(19) United States
(12) Patent Application Publication
(10) Pub. No.: US 2023/0236188 A1
(43) Pub. Date: Jul. 27, 2023

(54) METHODS OF DIAGNOSING AND TREATING LUPUS

(71) Applicant: BRISTOL-MYERS SQUIBB COMPANY, Princeston, NJ (US)

(72) Inventors: Laurence Celine Menard, Princeton, NJ (US); Steven G. Nadler, Princeton, NJ (US)

(21) Appl. No.: 18/152,056

(22) Filed: Jan. 9, 2023

Related U.S. Application Data
(63) Continuation of application No. 16/085,007, filed on Sep. 14, 2018, now abandoned.
(60) Provisional application No. 62/309,290, filed on Mar. 16, 2016.

(51) Int. Cl. G01N 33/564

(52) U.S. Cl. CPC G01N 33/564; G01N 2333/70532; G01N 2333/70578; G01N 2800/104; G01N 2333/70596; G01N 2333/70521; G01N 2333/70575; G01N 2800/52; G01N 2800/60

(57) ABSTRACT

In certain embodiments, the present invention provides a method of treating or preventing lupus (e.g., SLE) in a subject, comprising: (a) identifying the subject as having at least one differentially regulated biomarker selected from CD40, CD40L, CD86, CD80, and PD1; and (b) administering an agent that inhibits the CD40 or CD28 signaling pathway, thereby treating or preventing lupus in the subject. In other embodiments, the present invention provides a method of treating or preventing lupus (e.g., SLE) in a subject, comprising: (a) administering an agent that inhibits the CD40 or CD28 signaling pathway; (b) determining whether the agent neutralizes at least one differentially regulated biomarker selected from CD40, CD40L, CD86, CD80, and PD1; and (c) adjusting the dosing of the agent in the subject, thereby treating or preventing lupus in the subject.

### REFERENCE TO RELATED APPLICATIONS

This application is a continuation of 371 PCT application Ser. No. 16/085,007 filed Mar. 15, 2017, which is the national phase filing in the U.S. of PCT/US2017/022496 filed Mar. 15, 2017, which claims benefit to U.S. Provisional Application No. 62/309,290 filed Mar. 16, 2016, which is hereby incorporated in its entirety for all purposes.

### BACKGROUND OF THE INVENTION

Lupus is a group of conditions with similar underlying mechanisms involving autoimmunity. In these conditions, antibodies created by the body to attack antigens (e.g., viruses, bacteria) become unable to differentiate between antigens and healthy tissue. Thus, these antibodies begin to attack the body's own healthy tissues. Lupus is generally a chronic disease in which the signs and symptoms tend to come and go. Lupus also increases the risk of developing various other diseases such as heart disease, osteoporosis, and kidney disease. Types of lupus include, for example, systemic lupus erythematosus (SLE), cutaneous lupus erythematosus (CLE) (CLE includes, e.g., acute cutaneous lupus erythematosus (ACLE), subacute cutaneous lupus erythematosus (SCLE), intermittent cutaneous lupus erythematosus, and chronic cutaneous lupus), drug-induced lupus, and neonatal lupus. About 70% of all cases of lupus are SLE.

Diagnosing and monitoring of lupus remain problematic. Thus, the need exists for novel ways of identifying, assessing, and treating individuals affected by the disease.

### BRIEF SUMMARY OF THE INVENTION

In certain embodiments, the present invention provides a method of treating or preventing lupus in a subject, comprising: (a) identifying the subject as having at least one differentially regulated biomarker selected from CD40, CD40L, CD86, CD80, and PD1; and (b) administering an agent that inhibits the CD40 or CD28 signaling pathway, thereby treating or preventing lupus in the subject. For example, the lupus is systemic lupus erythematosus (SLE).

In certain specific embodiments, the method of the present invention comprises identifying the subject as having at least two, at least three, or at least four, differentially regulated biomarker selected from CD40, CD40L, CD86, CD80, and PD1. To illustrate, the differentially regulated biomarker comprises down-regulated expression of CD40, up-regulated expression of CD40L, up-regulated expression of CD86, up-regulated expression of CD80, and/or up-regulated expression of PD1.

In certain specific embodiments, the method of the present invention administering an agent that specifically binds to CD40 (e.g., an anti-CD40 antibody). Optionally, the agent is an anti-CD40 domain antibody (e.g., BMS-986090). In certain specific embodiments, the method of the present invention administering an agent that specifically binds to CD40L (e.g., an anti-CD40L antibody). Optionally, the agent is an anti-CD40L domain antibody (e.g., BMS-986004). In certain specific embodiments, the method of the present invention administering an agent that specifically binds to CD28 (e.g., an anti-CD28 antibody). Optionally, the agent is an anti-CD28 domain antibody (e.g., BMS-931699).

In certain specific embodiments, the differentially regulated biomarker is detected in a whole blood sample of the subject. For example, the expression level (mRNA or protein) of the differentially regulated biomarker is detected. To illustrate, the differentially regulated biomarker is detected by a method comprising contacting a sample from the subject with an antibody which binds to the biomarker. In a specific embodiment, the subject is an African American.

In other embodiments, the present invention provides a method of treating or preventing lupus in a subject, comprising: (a) administering an agent that inhibits the CD40 or CD28 signaling pathway; (b) determining whether the agent neutralizes at least one differentially regulated biomarker selected from CD40, CD40L, CD86, CD80, and PD1; and (c) adjusting the dosing of the agent in the subject, thereby treating or preventing lupus in the subject. For example, the lupus is systemic lupus erythematosus (SLE).

In certain specific embodiments, the method of the present invention comprises determining whether the agent neutralizes at least two, at least three, or at least four, differentially regulated biomarker selected from CD40, CD40L, CD86, CD80, and PD1. To illustrate, the differentially regulated biomarker comprises down-regulated expression of CD40, up-regulated expression of CD40L, up-regulated expression of CD86, up-regulated expression of CD80, and/or up-regulated expression of PD1. For example, the agent neutralizes the differentially regulated biomarker by at least 10%, at least 20%, at least 30%, at least 40% or at least 50%.

In certain specific embodiments, the method of the present invention administering an agent that specifically binds to CD40 (e.g., an anti-CD40 antibody). Optionally, the agent is an anti-CD40 domain antibody (e.g., BMS-986090). In certain specific embodiments, the method of the present invention administering an agent that specifically binds to CD40L (e.g., an anti-CD40L antibody). Optionally, the agent is an anti-CD40L domain antibody (e.g., BMS-986004). In certain specific embodiments, the method of the present invention administering an agent that specifically binds to CD28 (e.g., an anti-CD28 antibody). Optionally, the agent is an anti-CD28 domain antibody (e.g., BMS-931699).

In certain specific embodiments, the differentially regulated biomarker is detected in a whole blood sample of the subject. For example, the expression level (mRNA or protein) of the differentially regulated biomarker is detected. To illustrate, the differentially regulated biomarker is detected by a method comprising contacting a sample from the subject with an antibody which binds to the biomarker. In a specific embodiment, the subject is an African American.

In other embodiments, the present invention provides a kit comprising: (1) an antibody which specifically binds to at least one differentially regulated biomarker selected from CD40, CD40L, CD86, CD80, and PD1; and (2) instructions for use of said kit. For example, the kit comprise at least two antibodies which specifically bind to at least two differentially regulated biomarker selected from CD40, CD40L, CD86, CD80, and PD1.

### BRIEF DESCRIPTION OF THE DRAWING

FIGS. 1A-1C show increased frequency of CD86+ B cells in African American (Afr. Am.) Systemic Lupus Erythematosus (SLE) patients. FIG. 1A is a representative zebra plot of CD86 and CD27 expression on CD19+ total B cells from peripheral blood mononuclear cells of a normal healthy volunteer (NHV) and SLE European American (Eur. Am.) and Afr. Am. patients. Numbers on zebra plots represent percentages of cells in each quadrant. FIGS. 1B and 1C show summarized frequencies of CD86+ CD27− B cells (FIG. 1B) or CD86+ CD27+ memory B cells (FIG. 1C) in 56 Eur. Am. and 13 Afr. Am. NHV donors and 39 Eur. Am. and 29 Afr. Am. SLE patients. The horizontal bars represent the average for each group. P values are indicated (Mann Whitney test), n.s.: non significant

FIGS. 2A-2C show higher frequencies of CD40 ligand (CD40L)+ B cells in African American (Afr. Am.) Systemic Lupus Erythematosus (SLE) patients. FIG. 1A is a representative zebra plot of CD40L expression on CD19+ CD27− B cells from peripheral blood mononuclear cells of a normal healthy volunteer (NHV) and European American (Eur. Am.) and Afr. Am. SLE patients. Numbers on zebra plots represent frequencies of CD40L+ CD27− B cells. FIGS. 2B and 2C show summarized frequencies of CD40L+ CD27− B cells (FIG. 2B) or CD40L+ CD27+ B cells (FIG. 2C) in 55 Eur. Am. and 13 Afr. Am. NHV donors and 34 Eur. Am. and 23 Afr. Am. SLE patients. The horizontal bars represent the average for each group. P values are indicated (Mann Whitney test), n.s.: non significant.

FIGS. 3A-3E show that African American (Afr. Am.) Systemic Lupus Erythematosus (SLE) patients express lower levels of surface CD40 on their B cells. FIG. 3A is a representative zebra plot of CD40 expression by CD19+CD27− B cells from peripheral blood mononuclear cells of a normal healthy volunteer (NHV) and European American (Eur. Am.) and Afr. Am. SLE patients. Numbers on zebra plots represent frequencies of B cells with low surface CD40 expression (CD40lo). FIGS. 3B and 3C show summarized frequencies of CD40lo CD27− B cells (FIG. 3B) and CD40lo CD27+ B cells (FIG. 3C) in 55 Eur. Am. and 13 Afr. Am. NHV donors and 34 Eur. Am. and 23 Afr. Am. SLE patients. The horizontal bars represent the average for each group. P values are indicated (Mann Whitney test), n.s.: non significant. FIGS. 3D and 3E show Spearman correlation between frequencies of CD40L+ CD27− B cells and CD40lo CD27− B cells in 23 Afr. Am. (FIG. 3D) and 34 Eur. Am. (FIG. 3E) patients. Spearman r and p values are indicated on each plot.

FIGS. 4A-4B show rapid down-regulation of surface CD40 in B cells activated by CD40 ligand (CD40L). FIG. 4A shows CD86 and CD40 expression in freshly isolated CD19+ B cells from a normal healthy volunteer (NHV) and after stimulation with soluble CD40L-isoleucine zipper (CD40L-IZ) or anti-IgM F(ab′)2 for 3 h and 24 h. FIG. 4B shows CD86 and CD40 expression in isolated CD19+ B cells from a NHV cultured for 1 h or 24 h in medium only or with CD40L-IZ or with 1% or 10% of CHO cells stably transfected with human CD40LG (hCD40L-CHO). Numbers on zebra plots represent percentages of cells in each quadrant. Experiments were performed at least twice, using two donors per experiment.

FIGS. 5A-5I show internalization of CD40 following engagement with CD40 ligand (CD40L). FIGS. 5A, 5B, and 5C show representative pictures of CD19, CD40, NF-kB and nuclear 7-aminoactinomycin (7-AAD) stainings in unstimulated B cells (FIG. 5A), B cells stimulated with soluble CD40L-isoleucine zipper (CD40L-IZ) (FIG. 5B) or CHO cells stably transfected with human CD40LG (hCD40L-CHO) (FIG. 5C) for 1 h. FIGS. 5D, 5E, and 5F show histograms representing CD40 internalization (FIG. 5D), CD45 internalization (FIG. 5E) and NF-kB nuclear translocation (defined as the similarity score between NF-kB and 7-Aminoactinomycin D (7-AAD) staining) (FIG. 5F) in unstimulated B cells (Unstim, grey), CD40L-IZ-stimulated B cells (red) or hCD40L-CHO cells-stimulated B cells (blue). FIGS. 5G, 5H, and 5I show internalization score of CD40 (red) and CD45 (grey) (FIG. 5G), percentage of cells with internalized CD40 (internalization score>2.5) (FIG. 5H) and percentage of B cells with p50 NF-kB nuclear translocation (NF-kB:7-AAD similarity score>0) (FIG. 5I). Averaged results from 2 donors from 4 different experiments are represented on the graphs. The horizontal bars represent the average of 4 experiments for each stimulation condition. *: p<0.05 by Mann Whitney test vs. unstimulated B cells (unstim) (FIGS. 5G-5I). Purified total B cells from normal healthy volunteers were used.

FIGS. 6A-6F show that B cell expression of CD40 ligand (CD40L) can induce CD40 internalization and pathway activation in trans. FIG. 6A shows CD86 and CD40L expression in freshly isolated CD19+ B cells and after 3 days of culture with CpG-oligodeoxynucleotides (CpG) or soluble CD40L-isoleucine zipper (CD40L-IZ). FIGS. 6B, 6C, 6D, 6E, and 6F show internalization of CD40 (FIG. 6B) and NF-kB nuclear translocation (NF-kB: 7-Aminoactinomycin D (7-AAD) similarity score) (FIG. 6C) on B cells freshly isolated (unstim) or co-cultured for 1 h with autologous B cells previously stimulated for 3 days with CD40L-IZ (CD40L-IZ stim B cells) or CpG (CpG-stim B cells). Quantification of CD40 (black) and CD45 (grey) internalization by median internalization score (FIG. 6D), % of cells with CD40 internalization score>2.5 (FIG. 6E) or % of cells with NF-kB translocation (NF-kB:7AAD similarity score>0) (FIG. 6F) on B cells stimulated in indicated conditions. Averaged results from 2 donors from 4 different experiments are represented on the graphs (FIGS. 6D-6F). *: p<0.05 by Mann Whitney test. Purified total B cells from normal healthy volunteers were used.

FIGS. 7A-7D show increased frequency of double negative (DN) B cells in African American (Afr. Am.) Systemic Lupus Erythematosus (SLE) patients. Frequencies of CD19+ IgD−CD27−DN B cells (FIG. 7A), CD19+IgD+CD27− naïve B cells (FIG. 7B), CD19+IgD+CD27+ unswitched memory B cells (FIG. 7C) and CD19+IgD−CD27+ switched B cells (FIG. 7D) in whole blood of 38 European American (Eur. Am.) and 11 African American (Afr. Am.) normal healthy volunteer (NHV) donors and 21 Eur. Am. and 21 Afr. Am. SLE patients. The horizontal bars represent the average for each group. P values are indicated (Mann Whitney test), n.s.: non significant.

FIGS. 8A-8D show that higher frequencies of CD40^{lo} CD27− B cells correlate with higher titers of autoantibodies Anti-Smith/ribonucleoprotein (Sm/RNP) (FIG. 8A), anti-Sm (FIG. 8B), anti-RNP-70 (FIG. 8C) and anti-dsDNA (FIG. 8D) IgG plasma levels in 15 European American (Eur. Am.) and 5 African American (Afr. Am.) Systemic Lupus Erythematosus (SLE) patients with low frequencies of CD40loCD27−B cells and 11 Eur. Am. and 15 Afr. Am. SLE patients with high frequencies of CD40loCD27−B cells (cut-off was set at 1.54% of CD40loCD27− B cells, which corresponds to the 90th percentile in normal healthy volunteer donors). P value for statistically significant differences are indicated (Mann Whitney), n.s.: non significant. The horizontal bar represents the average for each group.

FIGS. 9A-9D show increased frequency of CD80+ and PD1+ B cells in African American SLE patients. Frequencies of CD80+ CD19+ CD27− B cells (FIG. 9A), CD80+ CD19+ CD27+ B cells (FIG. 9B), PD1+ CD19+ CD27− B cells (FIG. 9C) and PD1+ CD19+ CD27+ B cells (FIG. 9D) in PBMC from African American (Afr. Am.) and European American (Eur. Am.) normal healthy volunteers (NHV) and SLE patients. 68 NHV and 68 SLE donors were used for CD80+ B cells frequencies and 62 NHV and 53 SLE donors for PD1+ B cell frequencies. P values are indicated (Mann Whitney test).

FIGS. 10A-10D show expression of CD40L by T cells of African American and

European American SLE patients Summary of frequencies of CD40L+ CD4+ CD45RO− naïve T cells (FIG. 10A), CD40L+ CD4+ CD45RO+ memory T cells (FIG. 10B), CD40L+ CD8+ CD45RO− naïve T cells (FIG. 10C) and CD40L+ CD8+ CD45RO+ memory T cells (FIG. 10D) in PBMC from 67 normal healthy volunteers (NHV) and 52 SLE patients. P values when statistically significant are indicated (Mann Whitney test).

FIG. 11 shows plasma levels of soluble CD40L (sCD40L) in African American and European American NHV and SLE patients. sCD40L was measured by ELISA in plasma from 52 Eur. Am. and 4 Afr. Am NHV, and 36 Eur. Am. and 28 Afr. Am. SLE donors. P values when statistically significant are indicated (Mann Whitney test).

FIGS. 12A-12B show that stimulation with CD40 induces CD40^{lo}, CD86+ and PD1+ CD27− B cells with different kinetics. Induction of CD40^{lo}, CD86+ and PD1+ CD27− B cells by CD40L-IZ (FIG. 12A) and by anti-IgMF(ab′)2 (FIG. 12B) stimulation at 3 h, 24 h, 48 h.

FIG. 13 shows that CD40L-IZ does not prevent binding of CD40-PE to CD40. Cells were stained at 40C with anti-CD40PE without or with CD40L-IZ, washed and stimulated at 370C with CD40L. Internalization score and percentages of cells with high internalization of CD40 (score>2.5) were similar whether staining with CD40-PE antibody was performed with or without CD40L-IZ.

FIG. 14 shows that gating strategy for B cell subsets excludes doublets and CD3+ cells. Flow cytometry dot plots showing a representative gating strategy for whole blood B cell subsets. Single cells are selected, then CD3+ are excluded from the CD19+ gate. IgD and CD27 expressions are used to gate for naive, double negative (DN), switched and unswitched memory B cells in the CD19+ gate.

FIG. 15 shows increased CD86 expression in both IgD+ and IgD− CD27− B cells in African American SLE patients compared to patients of European descent. Summary of frequencies of CD86+ IgD+ CD27− (naïve) and CD86+ IgD−CD27− (DN) B cells in 21 African American (Afr. Am.) and 21 European American(Eur. Am.) SLE patients. p-values by Mann Whitney test are indicated.

FIGS. 16A-16B show that glucocorticoid (GC) use is not associated with a higher frequency of CD40L+ CD27− B cells. FIG. 16A shows percentages of CD40L+CD27− B cells in 34 European American (Eur. Am.) and 23 African American (Afr. Am.) patients, treated (GC) or not treated (no GC) with glucocorticoids. FIG. 16B shows percentages of CD40L+CD27− B cells and GC dose (mg/day) in 36 treated patients show no significant correlation (Spearman correlation).

FIGS. 17A-17B show that recent flares do not account for the observed activated B cell phenotype. FIG. 17A shows Spearman correlation of % CD86+CD27− B cells and SLEDAI-2k in 25 African American SLE patients. Spearman r and p-value are indicated on the plot. The dotted line represents the 2.5% threshold. FIG. 17B shows percentages of CD86+ CD27− B cells in African American (Afr. Am.) or European American (Eur. Am.) patients who flared less (10 Eur. Am. and 8 Afr. Am.) or more (29 Eur. Am and 22 Afr. Am.) than 3 months ago.

FIG. 18 shows that B cells from African American (Afr. Am.) and European American (Eur. Am.) systemic lupus erythematosus (SLE) patients and from normal healthy volunteers (NHV) respond similarly to CD40 ligand (CD40L) stimulation. CD86 median fluorescence intensity (MFI) was measured on B cells after overnight stimulation with CD40L isoleucine zipper of whole blood from 24 Eur. Am and Afr. Am. NHV, 19 Eur. Am. and 7 Afr. Am. SLE donors. Fold change of CD86 MFI in stimulated sample over non stimulated sample is represented.

FIG. 19 shows higher anti-Sm/RNP and anti-RNP70 IgG titers in African American patients. Autoantibody plasma (IgG, U/ml) levels in 27 African American and 31 European American SLE patients. P-value for statistically significant differences are indicated (Mann Whitney).

### DETAILED DESCRIPTION OF THE INVENTION

Lupus is an autoimmune disease that results in multi-organ involvement. This anti-self response in SLE patients is characterized by autoantibodies directed against a variety of nuclear and cytoplasmic cellular components. These autoantibodies bind to their respective antigens, forming immune complexes that circulate and eventually deposit in tissues. This immune complex deposition causes chronic inflammation and tissue damage.

Diagnosing and monitoring disease activity are problematic in patients with lupus. Diagnosis is problematic because the spectrum of disease is broad and ranges from subtle or vague symptoms to life-threatening multi-organ failure. There also are other diseases with multi-system involvement that can be mistaken for lupus, and vice versa. Monitoring disease activity also is problematic in caring for patients with lupus. Lupus progresses in a series of flares, or periods of acute illness, followed by remissions. The symptoms of a flare, which vary considerably between patients and even within the same patient, include malaise, fever, symmetric joint pain, and photosensitivity (development of rashes after brief sun exposure). Other symptoms of lupus include hair loss, ulcers of mucous membranes and inflammation of the lining of the heart and lungs, which leads to chest pain. Red blood cells, platelets and white blood cells can be targeted in lupus, resulting in anemia and bleeding problems. More seriously, immune complex deposition and chronic inflammation in the blood vessels can lead to kidney involvement and occasionally kidney failure, requiring dialysis or kidney transplantation. Since the blood vessel is a major target of the autoimmune response in lupus, premature strokes and heart disease are not uncommon. Over time, however, these flares can lead to irreversible organ damage.

Systemic Lupus Erythematosus (SLE) is a complex systemic autoimmune disease driven by both innate and adaptive immune cells. African Americans tend to present with more severe disease at an earlier age compared to patients of European ancestry. In order to better understand the immunological differences between African American and European American patients, Applicants analyzed the frequencies of B cell subsets and the expression of B cell activation markers from a total of 72 SLE patients and 69 normal healthy volunteers. Applicants found that B cells expressing the activation markers CD86, CD80, PD1 and CD40L, as well as CD19+CD27−IgD− double negative B cells, were enriched in African American patients vs. patients of European ancestry. In addition to increased expression of CD40L, surface levels of CD40 on B cells were lower, suggesting the engagement of the CD40 pathway. In vitro experiments confirmed that CD40L expressed by B cells could lead to CD40 activation and internalization on adjacent B cells. Thus, Applicants' findings help the development of novel diagnostics and therapies for lupus.

In certain embodiments, the present invention provides a method of treating or preventing lupus (e.g., SLE) in a subject, comprising: (a) identifying the subject as having at least one differentially regulated biomarker selected from CD40, CD40L, CD86, CD80, and PD1; and (b) administering an agent that inhibits the CD40 or CD28 signaling pathway, thereby treating or preventing lupus in the subject. In certain specific embodiments, the method of the present invention comprises identifying the subject as having at least two, at least three, or at least four, differentially regulated biomarker selected from CD40, CD40L, CD86, CD80, and PD1. To illustrate, the differentially regulated biomarker comprises down-regulated expression of CD40, up-regulated expression of CD40L, up-regulated expression of CD86, up-regulated expression of CD80, and/or up-regulated expression of PD1. In certain specific embodiments, the method of the present invention administering an agent that specifically binds to CD40 (e.g., an anti-CD40 antibody). Optionally, the agent is an anti-CD40 domain antibody (e.g., BMS-986090). In certain specific embodiments, the method of the present invention administering an agent that specifically binds to CD40L (e.g., an anti-CD40L antibody). Optionally, the agent is an anti-CD40L domain antibody (e.g., BMS-986004). In certain specific embodiments, the method of the present invention administering an agent that specifically binds to CD28 (e.g., an anti-CD28 antibody). Optionally, the agent is an anti-CD28 domain antibody (e.g., BMS-931699). In certain specific embodiments, the differentially regulated biomarker is detected in a whole blood sample of the subject. For example, the expression level (mRNA or protein) of the differentially regulated biomarker is detected. To illustrate, the differentially regulated biomarker is detected by a method comprising contacting a sample from the subject with an antibody which binds to the biomarker. In a specific embodiment, the subject is an African American.

In other embodiments, the present invention provides a method of treating or preventing lupus in a subject, comprising: (a) administering an agent that inhibits the CD40 or CD28 signaling pathway; (b) determining whether the agent neutralizes at least one differentially regulated biomarker selected from CD40, CD40L, CD86, CD80, and PD1; and (c) adjusting the dosing of the agent in the subject, thereby treating or preventing lupus in the subject. In certain specific embodiments, the method of the present invention comprises determining whether the agent neutralizes at least two, at least three, or at least four, differentially regulated biomarker selected from CD40, CD40L, CD86, CD80, and PD1. To illustrate, the differentially regulated biomarker comprises down-regulated expression of CD40, up-regulated expression of CD40L, up-regulated expression of CD86, up-regulated expression of CD80, and/or up-regulated expression of PD1. For example, the agent neutralizes the differentially regulated biomarker by at least 10%, at least 20%, at least 30%, at least 40% or at least 50%. In certain specific embodiments, the method of the present invention administering an agent that specifically binds to CD40 (e.g., an anti-CD40 antibody). Optionally, the agent is an anti-CD40 domain antibody (e.g., BMS-986090). In certain specific embodiments, the method of the present invention administering an agent that specifically binds to CD40L (e.g., an anti-CD40L antibody). Optionally, the agent is an anti-CD40L domain antibody (e.g., BMS-986004). In certain specific embodiments, the method of the present invention administering an agent that specifically binds to CD28 (e.g., an anti-CD28 antibody). Optionally, the agent is an anti-CD28 domain antibody (e.g., BMS-931699). In certain specific embodiments, the differentially regulated biomarker is detected in a whole blood sample of the subject. For example, the expression level (mRNA or protein) of the differentially regulated biomarker is detected. To illustrate, the differentially regulated biomarker is detected by a method comprising contacting a sample from the subject with an antibody which binds to the biomarker. In a specific embodiment, the subject is an African American.

In other embodiments, the present invention provides a kit comprising: (1) an antibody which specifically binds to at least one differentially regulated biomarker selected from CD40, CD40L, CD86, CD80, and PD1; and (2) instructions for use of said kit. For example, the kit comprise at least two antibodies which specifically bind to at least two differentially regulated biomarker selected from CD40, CD40L, CD86, CD80, and PD1.

### Definitions

As used herein, each of the following terms has the meaning associated with it in this section.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The term “differentially regulated biomarker”, “differentially expressed biomarker” as described herein (e.g., CD40, CD40L, CD86, CD80, and PD1) refers to an increase or decrease in the expression level of a biomarker in a test sample, such as a sample derived from a patient suffering from lupus that is greater or less than the standard error of the assay employed to assess expression. For example, the alteration can be at least twice or more times greater than or less than the expression level of the biomarkers in a control sample (e.g., a sample from a healthy subject not having the associated disease), or the average expression level in several control samples. The altered expression of a biomarker can be determined at the protein or nucleic acid (e.g., mRNA) level.

A “biomarker” or “marker” is a gene, mRNA, or protein that undergoes alterations in expression that are associated with progression of lupus or responsiveness to treatment. The alteration can be in amount and/or activity in a biological sample (e.g., a blood, plasma, urine or a serum sample) obtained from a subject having lupus, as compared to its amount and/or activity, in a biological sample obtained from a baseline or prior value for the subject, the subject at a different time interval, an average or median value for a lupus patient population, a healthy control, or a healthy subject population (e.g., a control); such alterations in expression and/or activity are associated with progression of a disease state, such as lupus. For example, a marker of the invention which is associated with progression of lupus or predictive of responsiveness to therapeutics can have an altered expression level, protein level, or protein activity, in a biological sample obtained from a subject having, or suspected of having, lupus as compared to a biological sample obtained from a control subject.

A “nucleic acid” “marker” or “biomarker” is a nucleic acid (e.g., DNA, mRNA, cDNA) encoded by or corresponding to a marker as described herein. For example, such marker nucleic acid molecules include DNA (e.g. , genomic DNA and cDNA) comprising the entire or a partial sequence of any of the nucleic acid sequences set forth, or the complement or hybridizing fragment of such a sequence. The marker nucleic acid molecules also include RNA comprising the entire or a partial sequence of any of the nucleic acid sequences set forth herein, or the complement of such a sequence, wherein all thymidine residues are replaced with uridine residues. A “marker protein” is a protein encoded by or corresponding to a marker of the invention. A marker protein comprises the entire or a partial sequence of a protein encoded by any of the sequences set forth herein, or a fragment thereof. The terms “protein” and “polypeptide” are used interchangeably herein.

As used herein, a “disease progression” includes a measure (e.g., one or more measures) of a worsening, stability, or improvement of one or more symptoms and/or disability in a subject. In certain embodiments, disease progression is evaluated as a steady worsening, stability, or improvement of one or more symptoms and/or disability over time, as opposed to a relapse, which is relatively short in duration. In certain embodiments, the disease progression value is acquired in a subject with lupus (e.g., a subject with SLE, CLE, ACLE, SCLE, intermittent cutaneous lupus erythematosus, chronic cutaneous lupus, drug-induced lupus, or neonatal lupus).

Lupus is “treated,” “inhibited, “reduced,” or “prevented” if at least one symptom of the disease is reduced, alleviated, terminated, slowed, or prevented. As used herein, lupus is also “treated,” “inhibited,” or “reduced,” or “prevented,” if recurrence or relapse of the disease is reduced, slowed, delayed, or prevented. Exemplary clinical symptoms of lupus that can be used to aid in determining the disease status in a subject can include e.g., painful joints/arthralgia, fever of more than 100° F./38° C., arthritis/swollen joints, prolonged or extreme fatigue, skin rashes, anemia, kidney involvement, pain in the chest on deep breathing/pleurisy, butterfly-shaped rash across the cheeks and nose, sun or light sensitivity/photosensitivity, hair loss, blood clotting problems, Raynaud's phenomenon/fingers turning white and/or blue in the cold, seizures, mouth or nose ulcers, and any combination thereof. Clinical signs of lupus are routinely classified and standardized, e.g., using an SLEDAI rating system.

As used herein, the “Systemic Lupus Erythematosus Disease Activity Index” or “SLEDAI” is intended to have its customary meaning in the medical practice. EDSS is a rating system that is frequently used for classifying and standardizing MS. The accepted scores range from 0 (normal) to 105 (death due to lupus). A SLEDAI score of between 1-5 is indicative of mild disease activity in the subject; a SLEDAI score of between 6-10 is indicative of moderate disease activity in the subject; a SLEDAI score of between 11-19 is indicative of high disease activity in the subject; a SLEDAI score of 20-105 is indicative of very high disease activity in the subject.

“Responsiveness,” to “respond” to treatment, and other forms of this verb, as used herein, refer to the reaction of a subject to treatment with a lupus therapy. As an example, a subject responds to an lupus therapy if at least one symptom of lupus (e.g., disease progression) in the subject is reduced or retarded by about 10%, 20%, 30%, 40%, 50%, 60%, 70%, 80%, 90% or more. In another example, a subject responds to a lupus therapy, if at least one symptom of lupus in the subject is reduced by about 5%, 10%, 20%, 30%, 40%, 50% or more as determined by any appropriate measure, e.g., one or more of: a value of disease progression, a change in symptoms, and/or a modified SLEDAI value. In another example, a subject responds to treatment with a lupus therapy, if the subject has an increased time to progression. Several methods can be used to determine if a patient responds to a treatment including the assessments described herein, as set forth above.

An “overexpression,” “significantly higher level of expression,” or “upregulation” of the gene products refers to an expression level in a test sample that is greater than the standard error of the assay employed to assess the level of expression. In embodiments, the overexpression can be at least two, at least three, at least four, at least five, or at least ten or more times more than the expression level of the gene in a control sample or the average expression level of gene products in several control samples.

An “underexpression,” “significantly lower level of expression,” or “down-regulation” of the gene products refers to an expression level in a test sample that is lower than the standard error of the assay employed to assess the level of expression. In embodiments, the underexpression can be at least two, at least three, at least four, at least five, or at least ten or more times less than the expression level of the gene in a control sample or the average expression level of gene products in several control samples.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof.

Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VH, VL, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature, 341:544-546 (1989)), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., Science, 242:423-426 (1988); and Huston et al., Proc. Natl. Acad. Sci. USA, 85:5879-5883 (1988)). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic monomers.

“Sample,” “tissue sample,” “patient sample,” “patient cell or tissue sample” or “specimen” each refers to a biological sample obtained from a tissue or bodily fluid of a subject or patient. The source of the tissue sample can be solid tissue as from a fresh, frozen and/or preserved organ, tissue sample, biopsy, or aspirate; blood or any blood constituents (e.g., serum, plasma); bodily fluids such as urine, cerebral spinal fluid, whole blood, plasma and serum. The sample can include a non-cellular fraction (e.g., urine, plasma, serum, or other non-cellular body fluid). In one embodiment, the sample is a urine sample. In other embodiments, the body fluid from which the sample is obtained from an individual comprises blood (e.g., whole blood). In certain embodiments, the blood can be further processed to obtain plasma or serum. In another embodiment, the sample contains a tissue, cells (e.g., peripheral blood mononuclear cells (PBMC)). In one embodiment, the sample is a urine sample. For example, the sample can be a fine needle biopsy sample, an archival sample (e.g., an archived sample with a known diagnosis and/or treatment history), a histological section (e.g., a frozen or formalin-fixed section, e.g., after long term storage), among others. The term sample includes any material obtained and/or derived from a biological sample, including a polypeptide, and nucleic acid (e.g., genomic DNA, cDNA, RNA) purified or processed from the sample. Purification and/or processing of the sample can involve one or more of extraction, concentration, antibody isolation, sorting, concentration, fixation, addition of reagents and the like. The sample can contain compounds that are not naturally intermixed with the tissue in nature such as preservatives, anticoagulants, buffers, fixatives, nutrients, antibiotics or the like.

The amount of a biomarker, e.g., expression of gene products (e.g., one or more the biomarkers described herein), in a subject is “significantly” higher or lower than the normal amount of a marker, if the amount of the marker is greater or less, respectively, than the normal level by an amount greater than the standard error of the assay employed to assess amount, or at least two, three, four, five, ten or more times that amount. Alternatively, the amount of the marker in the subject can be considered “significantly” higher or lower than the normal amount if the amount is at least about 1.5, two, at least about three, at least about four, or at least about five times, higher or lower, respectively, than the normal amount of the marker.

### Methods of Detecting a Biomarker Protein

In certain embodiments, an antibody or antigen binding portion thereof can be used in a method for the detection of a differentially regulated biomarker protein (e.g., CD40, CD40L, CD86, CD80 or PD1) in a subject. For example, a body fluid (e.g., blood, serum or plasma) or tissue sample from the subject is contacted with an antibody or antigen binding portion thereof under conditions suitable for the formation of antibody-antigen complexes. The presence or amount of such complexes can then be determined by methods described herein and otherwise known in the art (see, e.g., O'Connor et al., Cancer Res., 48:1361-1366 (1988)), in which the presence or amount of complexes found in the test sample is compared to the presence or amount of complexes found in a series of standards or control samples containing a known amount of antigen. To illustrate, the method can employ an immunoassay, e.g., an enzyme immunoassay (EIA), enzyme-linked immunosorbent assay (ELISA), immunofluorescent assays, Western blotting, immunoelectrophoresis, fluid or gel precipitin reactions, immunodiffusion (single or double), radioimmunoassay (RIA), indirect competitive immunoassay, direct competitive immunoassay, non-competitive immunoassay, sandwich immunoassay, agglutination assay or other immunoassay describe herein and known in the art (see, e.g., Zola, Monoclonal Antibodies: A Manual of Techniques, pp. 147-158, CRC Press, Inc. (1987)). Immunoassays may be constructed in heterogeneous or homogeneous formats. Heterogeneous immunoassays are distinguished by incorporating a solid phase separation of bound analyte from free analyte or bound label from free label. Solid phases can take a variety of forms well known in the art, including but not limited to tubes, plates, beads, and strips. One particular form is the microtiter plate. The solid phase material may be comprised of a variety of glasses, polymers, plastics, papers, or membranes. Particularly desirable are plastics such as polystyrene. Heterogeneous immunoassays may be competitive or non-competitive (i.e., sandwich formats) (see, e.g., U.S. Pat. No. 7,195,882).

The antibody used for detecting the biomarker may be labeled. The label may be any detectable functionality that does not interfere with the binding of the antigen biomarker. Examples of suitable labels are those numerous labels known for use in immunoassay, including moieties that may be detected directly, such as fluorochrome, chemiluminscent, and radioactive labels, as well as moieties, such as enzymes, that must be reacted or derivatized to be detected. Examples of such labels include the radioisotopes 32P, 14C, 125I, 3H, and 131I, fluorophores such as rare-earth chelates or fluorescein and its derivatives, rhodamine and its derivatives, dansyl, umbelliferone, luceriferases, e.g., firefly luciferase and bacterial luciferase (see, e.g., U.S. Pat. No. 4,737,456), luciferin, 2,3-dihydrophthalazinediones, HRP, alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin (detectable by, e.g., avidin, streptavidin, streptavidin-HRP, and streptavidin-β-galactosidase with MUG), spin labels, bacteriophage labels, stable free radicals, and the like.

### Methods of Detecting a Biomarker Nucleic Acid

In certain embodiments, nucleic acids molecules which encode one or more differentially regulated biomarker nucleic acid (e.g., CD40, CD40L, CD86, CD80 or PD1) may be detected. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded; in certain embodiments the nucleic acid molecule is double-stranded DNA. Nucleic acid probes are sufficient for use as hybridization probes to identify nucleic acid molecules that correspond to a biomarker of the invention, e.g., those suitable for use as PCR primers for the amplification or mutation of nucleic acid molecules.

If so desired, a differentially regulated biomarker nucleic acid molecule can be isolated using standard molecular biology techniques and the sequence information in the database records described herein. Using all or a portion of such nucleic acid sequences, nucleic acid molecules can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989). A biomarker nucleic acid molecule can be amplified using cDNA, mRNA, or genomic DNA as a template and appropriate oligonucleotide primers according to standard PCR amplification techniques. The biomarker nucleic acid molecule so amplified can be cloned into an appropriate vector and characterized by DNA sequence analysis.

Furthermore, oligonucleotides (e.g., probes) corresponding to all or a portion of a nucleic acid molecule can be prepared by standard synthetic techniques, e.g., using an automated DNA synthesizer. Probes based on the sequence of a biomarker nucleic acid molecule can be used to detect transcripts (e.g., mRNA) or genomic sequences corresponding to one or more biomarkers of the invention. The probe comprises a label group attached thereto, e.g., a radioisotope, a fluorescent compound, an enzyme, or an enzyme co-factor. Such probes can be used as part of a diagnostic test kit for identifying cells or tissues which overexpress or underexpress the protein, such as by measuring levels of a nucleic acid molecule encoding the protein in a sample of cells from a subject.

### Kits

In certain embodiments, the present invention provides kits for detecting a biomarker in a biological sample (e.g., tissue, whole blood, serum, plasma, buccal scrape, saliva, cerebrospinal fluid, urine, stool, and bone marrow). For example, the kits comprise one or more antibodies (monoclonal or polyclonal) against one or more biomarkers (e.g., CD40, CD40L, CD86, CD80 or PD1), instructions for use of the kits, and optionally reagents necessary for facilitating an antibody-antigen complex formation and/or detection. The antibody may be labeled or unlabeled. Where the label is an enzyme, the kit will ordinarily include substrates and cofactors required by the enzyme, where the label is a fluorophore, a dye precursor that provides the detectable chromophore, and where the label is biotin, an avidin such as avidin, streptavidin, or streptavidin conjugated to HRP or β-galactosidase with MUG.

Such kits can be used to determine if a subject is suffering from or is at increased risk of developing lupus. Such kits can also be used for assessing the disease progression of a subject having lupus. Such kits can further be used for assessing a subject's response to a lupus therapy. Such kits can also be used for selecting or adjusting a dosing of a lupus therapy.

### Therapeutic Methods

In certain aspects, methods of the present invention can be used for selecting a subject suitable for a lupus therapy, for assessing the disease progression of a subject having lupus, for assessing a subject's response to a lupus therapy, and/or for selecting or adjusting a dosing of a lupus therapy.

In one specific embodiment, the invention provides novel and effective methods of treating lupus in a subject. In one specific embodiment, the method comprises: (a) identifying the subject as having at least one differentially regulated biomarker selected from CD40, CD40L, CD86, CD80, and PD1; and (b) administering an agent that inhibits the CD40 or CD28 signaling pathway, thereby treating or preventing lupus in the subject. In another specific embodiment, the method comprises: (a) administering an agent that inhibits the CD40 or CD28 signaling pathway; (b) determining whether the agent neutralizes at least one differentially regulated biomarker selected from CD40, CD40L, CD86, CD80, and PD1; and (c) adjusting the dosing of the agent in the subject, thereby treating or preventing lupus in the subject. In the methods of the invention, one or more of the biomarkers in a sample can be detected by any of assays as described above.

The term “treating” includes the administration of an agent to prevent or delay the onset of the symptoms, complications, or biochemical indicia of lupus, alleviating the symptoms or arresting or inhibiting further development of the disease. Treatment may be prophylactic (to prevent or delay the onset of the disease, or to prevent the manifestation of clinical or subclinical symptoms thereof) or therapeutic suppression or alleviation of symptoms after the manifestation of the disease.

The term “dosage,” “dose,” or “dosing” as used herein interchangeably, refers to an amount of a therapeutic agent which is administered to a subject having lupus.

The term “therapeutically effective dosage/dose/dosing,” as used herein, refers to an amount of a therapeutic agent which preferably results in a decrease in severity of disease symptoms, an increase in frequency and duration of disease symptom-free periods, or a prevention of impairment or disability due to the disease affliction. One of ordinary skill in the art would be able to determine such amounts based on such factors as the subject's size, the severity of the subject's symptoms, and the particular composition or route of administration selected.

There are several therapeutic agents presently used to modify the course of lupus. Such agents include, but are not limited to, nonsteroidal anti-inflammatory drugs (NSAID); antimalarials (e.g., hydroxychloroquine); corticosteroids (e.g., glucocorticoids); immunosuppressants (e.g., azathioprine, mycophenolate mofetil, or methotrexate); intravenous immunoglobulins; and a monoclonal antibody such as belimumab.

In certain embodiments, the methods of the invention provide the use of alternative therapies for the treatment of lupus. Such agents include, but are not limited to, an anti-CD40L antibody, an anti-CD40 antibody, and an anti-CD28 antibody. For example, an anti-CD40L antibody is a domain antibody which binds to and antagonize the CD40L activity, such as BMS-986004. BMS-986004 and uses thereof are disclosed in, e.g., WO 2013/056068, WO 2015/143209, and PCT/US2015/049338 (referred to therein as BMS2h-572-633-Fc fusion having the sequence of SEQ ID NO: 1355), the content of which is expressly incorporated by reference. For example, an anti-CD40 antibody is a domain antibody which binds to and antagonize the CD40 activity, such as BMS-986090. BMS-986090 and uses thereof are disclosed in, e.g., WO 2012/145673 and WO 2015/134988 (referred to therein as BMS3h-56-269-Fc fusion having the sequence of SEQ ID NO: 1287), the content of which is expressly incorporated by reference. For example, an anti-CD28 antibody is a domain antibody which binds to and antagonize the CD28 activity, such as BMS-931699. BMS-931699 and uses thereof are disclosed in, e.g., WO 2010/009391 and PCT/US2015/053233 (referred to therein as pegylated Bms1h-239-891 (D70C) having the sequence of SEQ ID NO: 543), the content of which is expressly incorporated by reference.

The present disclosure is further illustrated by the following examples, which should not be construed as further limiting. The contents of all figures and all references, patents and published patent applications cited throughout this application are expressly incorporated herein by reference in their entireties.

### Example 1

### Introduction

Systemic Lupus Erythematosus (SLE) is a complex systemic disease that can affect multiple organs. Both innate and adaptive immune cells are involved in driving the disease [1]. In particular B cells and autoantibody production are believed to participate in the pathogenesis of SLE. Indeed, SLE is characterized by the presence of anti-nuclear antibodies (ANA), anti-dsDNA, anti-Smith antigen (Sm) or anti-ribonucleoprotein (RNP) antibodies and disease activity and flares have been associated with the expansion of antibody-secreting cells [2].

SLE presentation varies greatly depending on the ancestral background. Compared to European Americans, African Americans are at higher risk of developing SLE and tend to be diagnosed earlier and suffer from a more severe disease with a higher rate of flares and progression to lupus nephritis (LN) and increased risk of death due to LN-related end-stage-renal disease. Although these disparities can be explained by the genetic background at disease onset, other factors such as poor socio-economic status, lack of social support or lower access to healthcare are major contributors to the accelerated and more severe course of disease [3-6]. Little is known about the immunological mechanisms of SLE that could account for the variations in susceptibility and severity in different ethnic groups. African American and Hispanics with moderate-to-severe active SLE showed a better response to rituximab in a phase II/III trial [7]. Also, a trend to better response with rituximab was seen in African American patients with LN [8]. These data suggest a B-cell-driven disease in these ethnic groups and imply that patients of different ancestries may respond differentially to treatments. In order to better understand mechanisms of disease and how they could be impacted by ancestral backgrounds, Applicants analyzed the B cell compartment of African American and European American SLE patients and healthy volunteer controls. Applicants discovered a distinct activated B cell signature in African American SLE patients with expansion of CD19+IgD−CD27− double negative (DN) B cells, higher expression of CD86 and CD40 ligand (CD40L) and lower CD40 surface expression in B cells, suggestive of a constitutively active CD40 pathway in these patients.

### Results

Activated Phenotype of B cells from African American SLE Patients

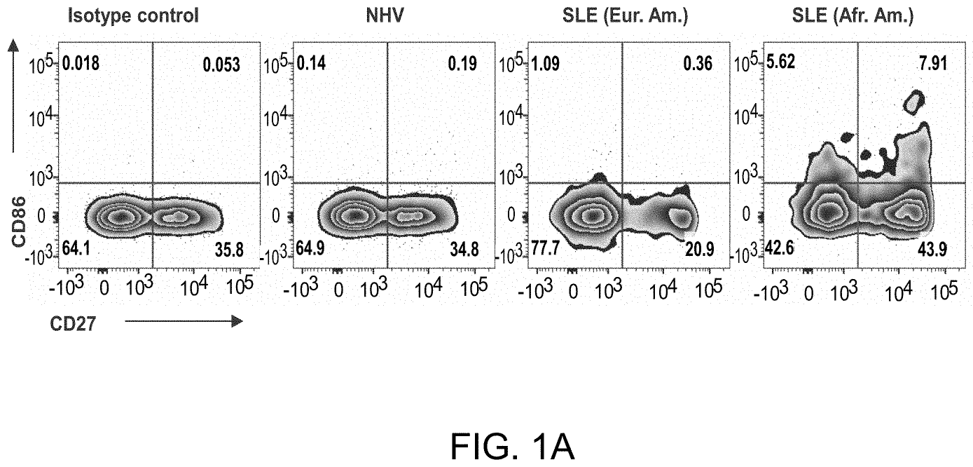

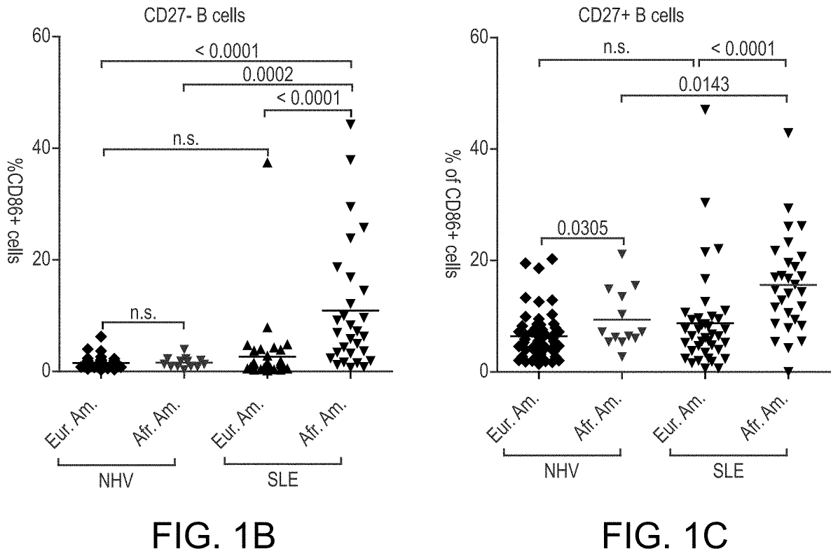

Applicants analyzed the expression of activation markers on B cells on 69 normal healthy volunteers (NHV) and 68 SLE patients, self-reported as of either African or European ancestry. Disease activity, which was low to moderate, medications, except for glucocorticoid use (which was more prevalent in the African American group), and co-morbidities were similar in the 2 ancestry groups (Table 1). Increased expression of the co-stimulatory molecule CD86 by SLE B cells has been previously described [9]. Applicants found an increased frequency of CD86 expressing B cells, both in the CD27− and CD27+ compartments in African American patients (average percentages of CD86+ cells: 11% of CD27− B cells and 16% of CD27+ B cells), compared to NHV of either ancestry (average percentages of CD86+ cells: 1.5% of CD27− B cells and 6-9% of CD27+ B cells) or SLE patients of European ancestry (average percentages of CD86+ cells: 2.7% of CD27− B cells and 9% of CD27+ B cells) (FIG. 1). Surprisingly, there was no significant increase in the frequency of CD86+ B cells in SLE patients of European descent relative to NHV, suggesting that African American patients may largely account for the previously described increase in CD86 expression by B cells in SLE (FIG. 1).

TABLE 1
Clinical data

| | African | European |
|---|---|---|
| | Americans | Americans |
| | (n = 29) | (n = 39) |
| SLEDAI-2K, mean ± SD | 3.8 ± 2.6 | 3.4 ± 1.8 |
| Total ACR classification criteria, mean ± SD | 5.7 ± 1.2 | 5.3 ± 1.3 |
| Duration of disease (years), mean ± SD | 13.5 ± 10.0 | 16.9 ± 14.8 |
| Time since last flare (years), mean ± SD | 2,944.3 | 3.1 ± 4.2 |
| Co-morbidities | | |
| Nephritis, n(%) | 14(48) | 15(38) |
| Sjogren Syndrome, n(%) | 1(3.4) | 4(10) |
| Antiphospholipid syndrome, n(%) | 0 | 4(10) |
| Medications | | |
| Hydroxychloroquine, n(%) | 15(52) | 18(46) |
| Mycophenolate mofetil, n(%) | 10(34) | 14(36) |
| Belimumab, n(%) | 3(10.3) | 1(2-6) |
| Glucocorticoids, n(%) | 23(79) | 19(49) |

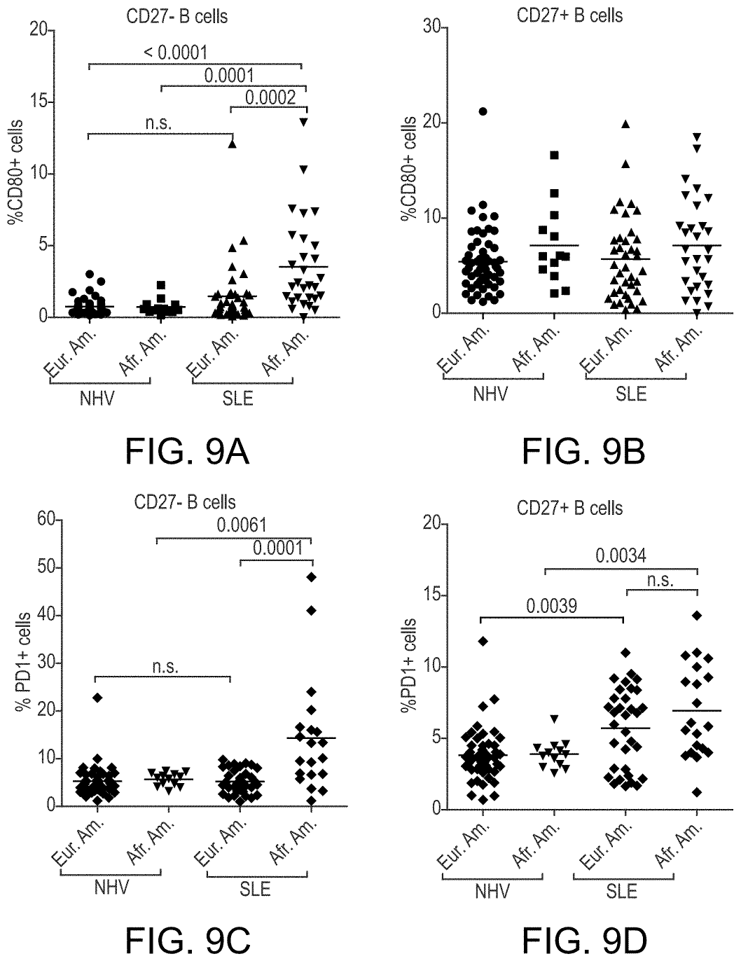

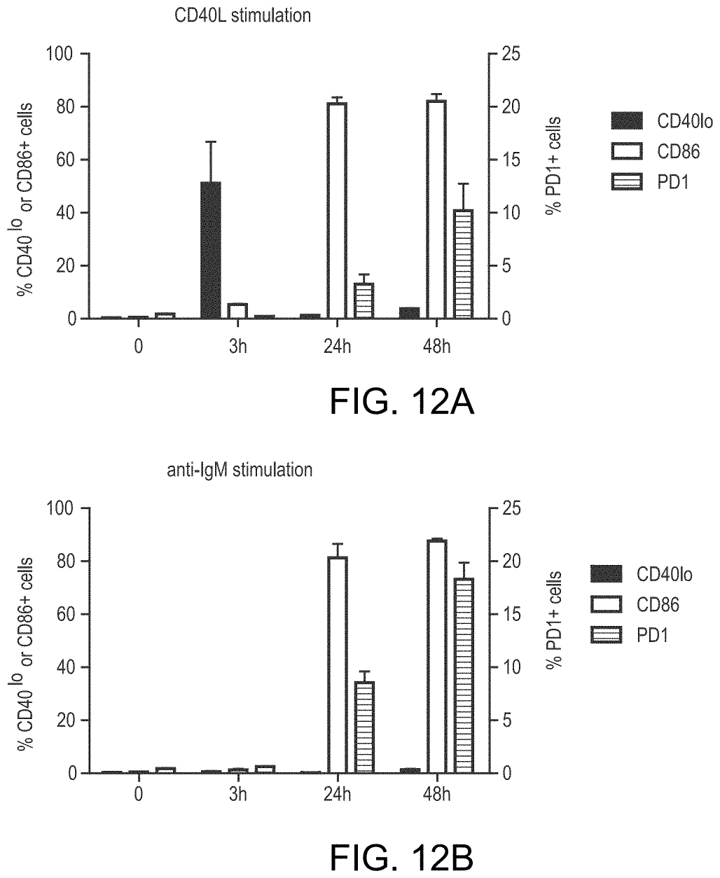

Applicants also analyzed the expression of CD80 and programmed cell death protein 1 (PD1), which are upregulated on B cells upon activation [10] (FIG. 12). Both CD80 and PD1 were significantly upregulated on CD27− B cells from African American SLE patients compared to European American SLE patients and all NHV groups (FIG. 9A and C). Interestingly, neither PD1 nor CD80 were upregulated in CD27− B cells from European American SLE patients compared to NHV. Finally, PD1 was upregulated in CD27+ memory B cells of both ancestral groups of SLE patients, compared to their respective NHV controls (FIG. 9D).

Increased CD40 Ligand (CD40L) and Decreased CD40 Surface Expressions on B Cells from African American SLE Patients

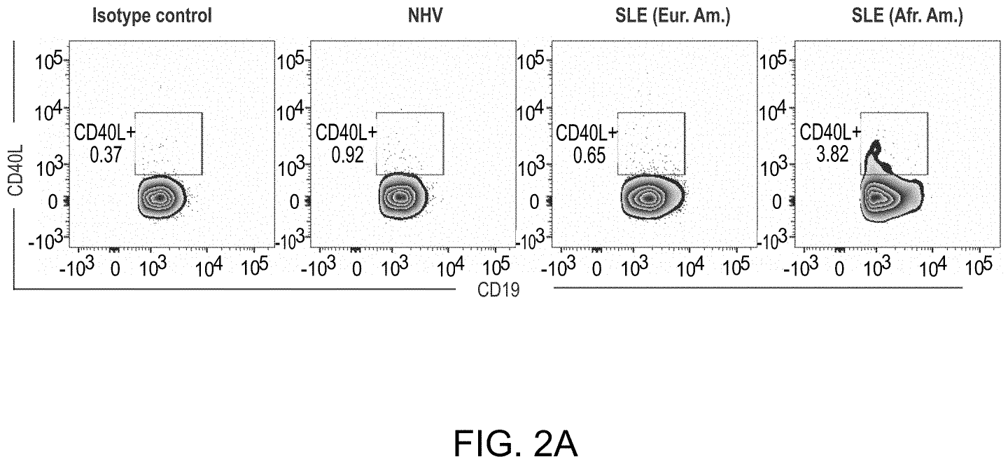

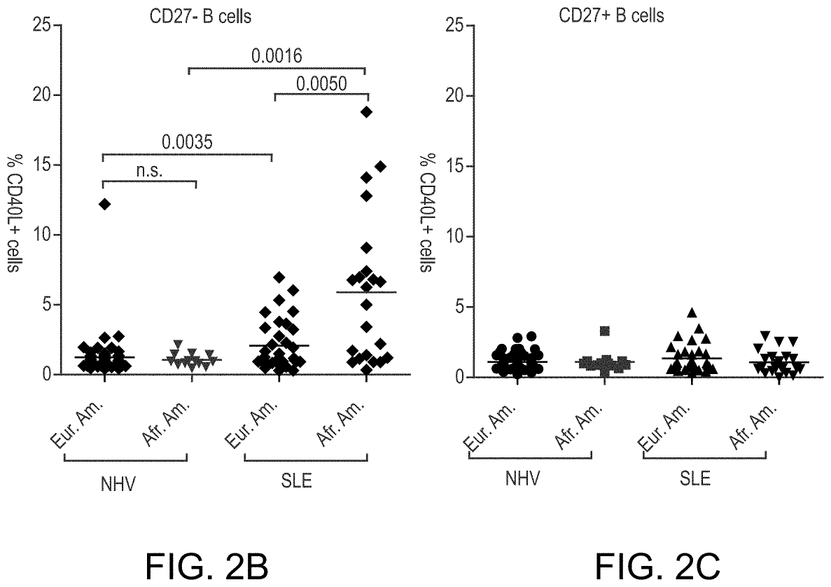

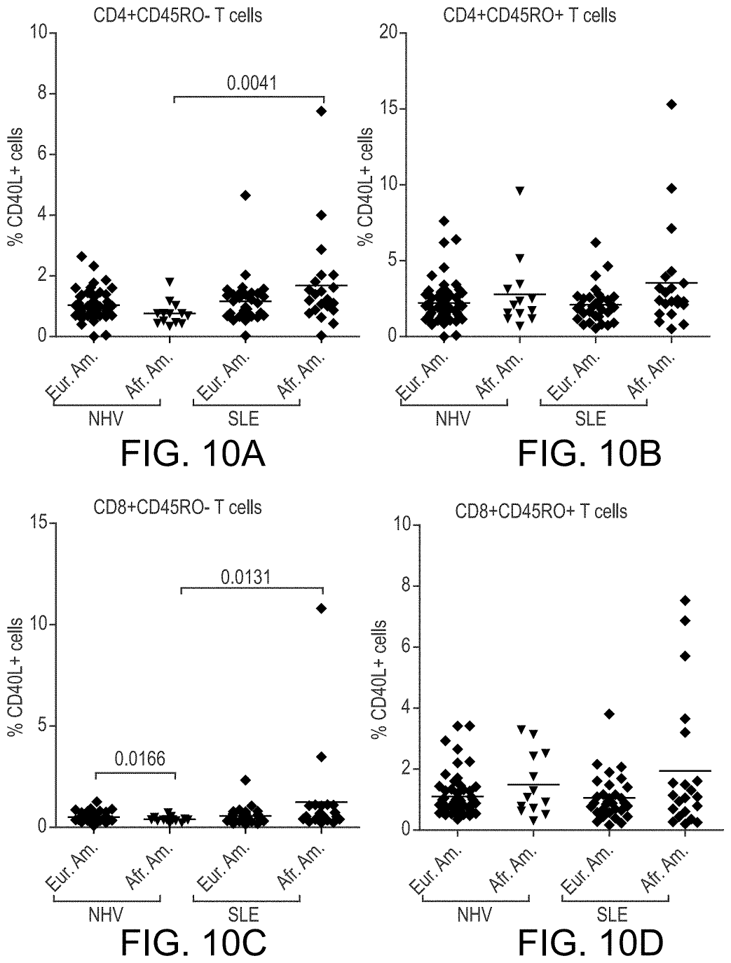

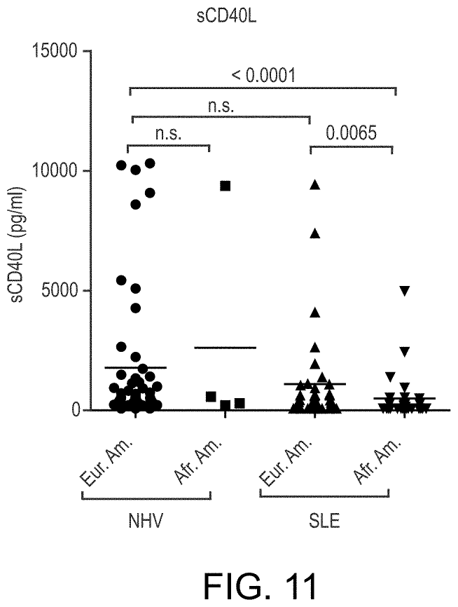

CD40L was shown to be increased in SLE T and B cells [11-13]. Applicants found increased expression of CD40L by CD27− B cells, not by CD27+ B cells, in our SLE cohort compared to NHV (FIG. 2). Moreover, the frequency of CD40L+ CD27− B cells was increased in African American SLE patients (average: 5.7% of CD40L+ CD27− B cells) compared to European American SLE patients (average: 2.1% of CD40L+ CD27− B cells, p<0.02). Analysis of CD40L expression on T cells revealed a modest but significant increase in African American SLE naïve CD45RO− CD4+ and CD45RO− CD8+ T cells compared to NHV (FIG. 10). CD40L can also be found in a soluble form (sCD40L) which is elevated in SLE and has the potential to activate B cells [14]. In this cohort, Applicants did not observe an increase in plasma levels of sCD40L in SLE patients. In fact, African American SLE patients showed reduced levels of sCD40L compared to European American NHV and SLE patients (FIG. 11).

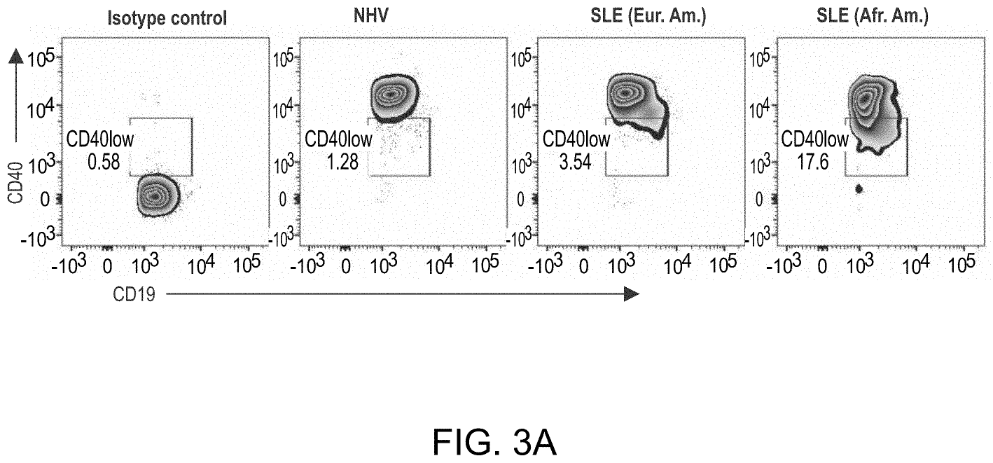

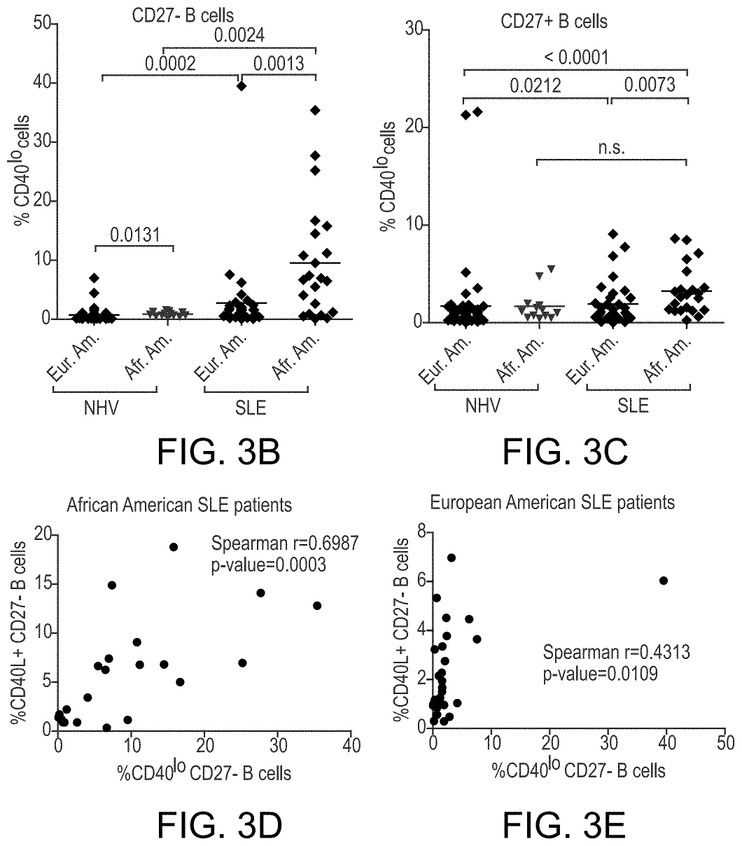

CD40, the receptor for CD40L, is constitutively expressed on B cells. Applicants observed that in some patients, a subset of B cells expressed lower levels of surface CD40 (‘CD40^{lo}’ B cells) (FIG. 3A). There was a major increase in the frequency of these CD40^{lo} CD27− B cells in African American SLE patients (average: 9.3% of CD40^{lo} CD27− B cells) compared to European American SLE patients (average: 2.8%, p<0.002) or African American NHV (average: 0.9%, p<0.005) (FIG. 3B). CD40^{lo} CD27− B cells were also increased in SLE patients of European descent (average: 2.8%) vs. NHV (average: 0.7%, p<0.0005), but to a lesser extent than in African American patients (FIG. 3B) Applicants observed a similar trend in CD27+ B cells, with slightly increased frequencies of CD40^{lo} CD27+ B cells in SLE patients vs. NHV of same ancestral background, and in African American (average: 3.1%) vs. European American SLE patients (average: 1.9%, p<0.02) (FIG. 3C).

Applicants then determined if the same patients harbored both CD40^{lo} B cells and CD40L+ B cells. There was a good correlation between the frequencies of CD40^{lo} B cells and CD40L+ B cells in African American patients (Spearman r=0.6987, p<0.0005) (FIG. 3D), which suggests the possibility of CD40−CD40L B-B cell interactions [15]. On the other hand, the correlation between frequencies of CD40^{lo} and CD40L+ B cells was weaker in European American patients (Spearman r=0.4313, p<0.02) (FIG. 3E). There was no correlation between the frequencies of CD40^{lo} B cells and the frequencies of CD40L+ CD4+CD45RO−T cells and CD40L+ CD8+CD45RO−T cells in SLE patients, independent of ancestry (Spearman r=0.084, p=0.55 and Spearman r=0.151, p=0.29 respectively). In addition, Applicants did not find an association between the lower plasma levels of sCD40L and higher frequencies of CD40^{lo} B cells in African Americans.

### CD40L Binding to CD40 Leads to CD40 Internalization

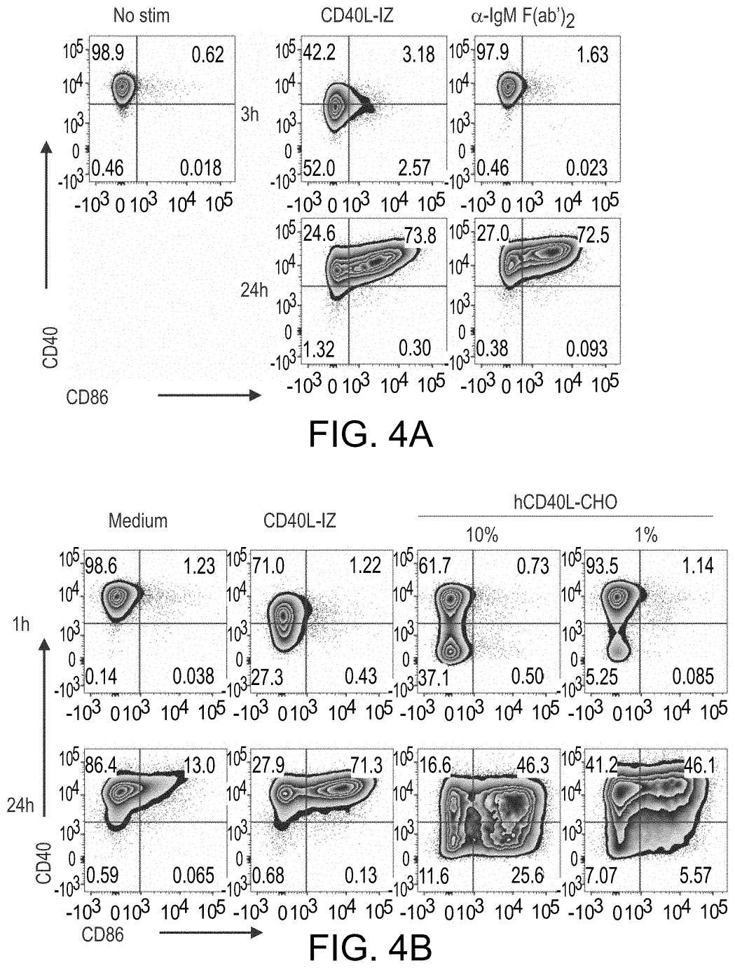

Engagement of CD40 on murine B cells by sCD40L leads to rapid loss of surface CD40 expression by receptor internalization [16-18]. To test whether CD40L expressed by B cells could engage CD40 on B cells and explain the phenotype observed in SLE African American patients, Applicants cultured purified B cells from NHV with soluble CD40L-isoleucine zipper (CD40L-IZ) or anti-IgM F(ab′)₂. Within 3 h, Applicants observed the appearance of ‘CD40^{lo}’ B cells in wells cultured with CD40L-IZ, but not with anti-IgM F(ab′)₂ stimulation. Expression of CD86 was upregulated by CD40L-IZ at 24 h, similar to what was seen with anti-IgM F(ab′)₂ (FIG. 4A)(FIG. 12), which confirms activation of B cells under both conditions.

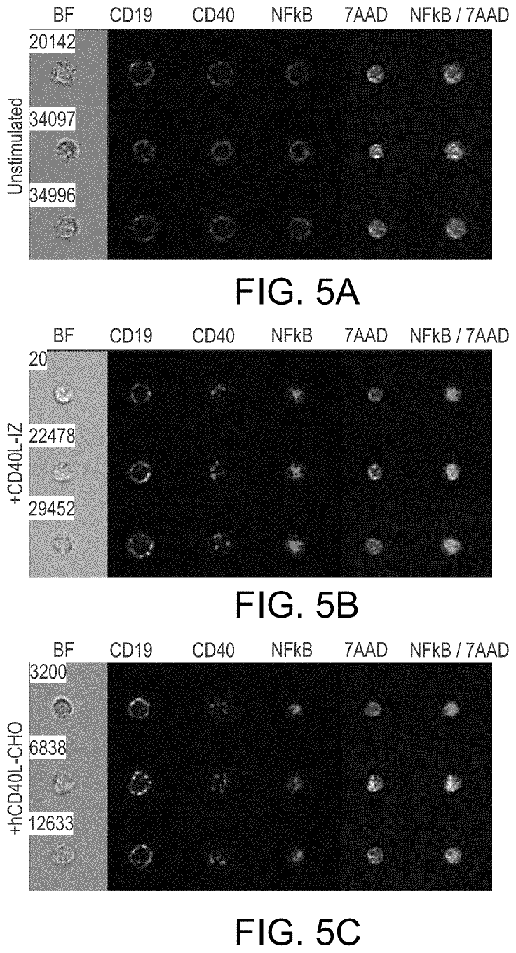

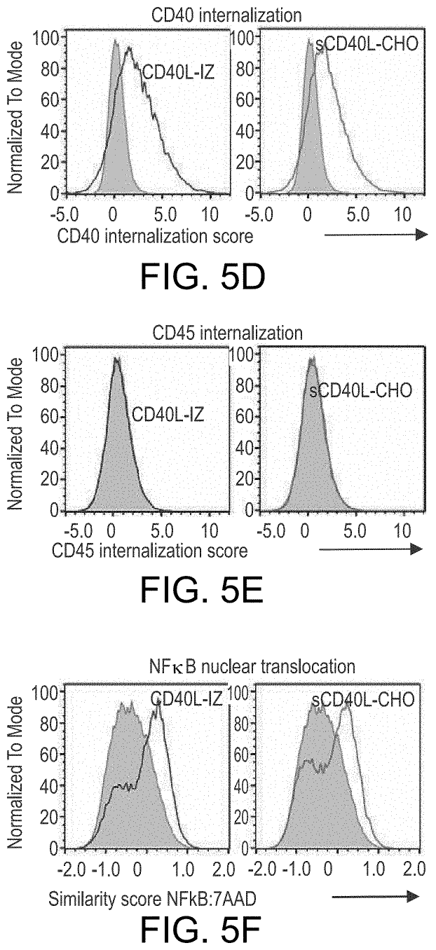

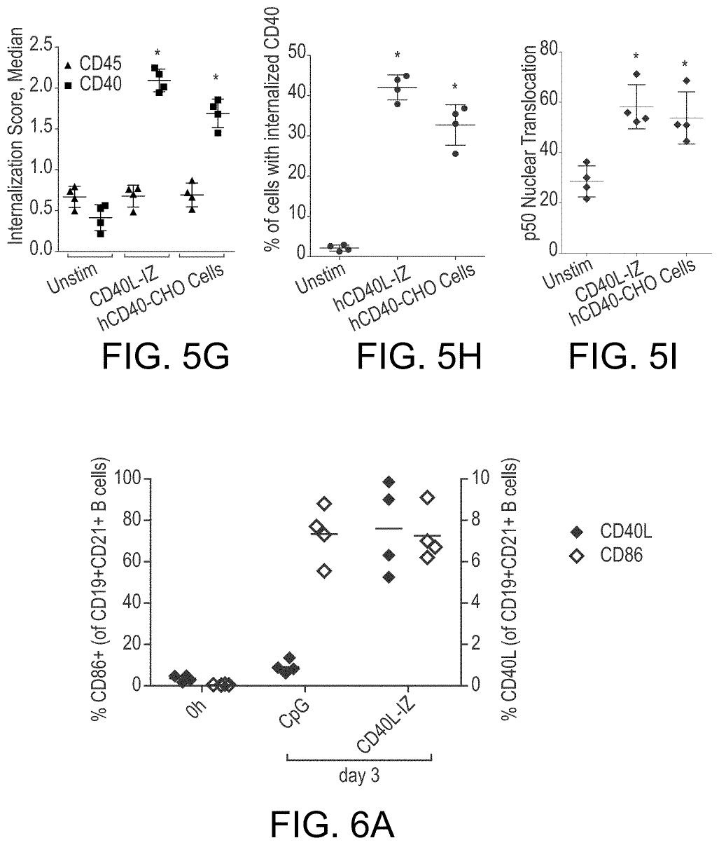

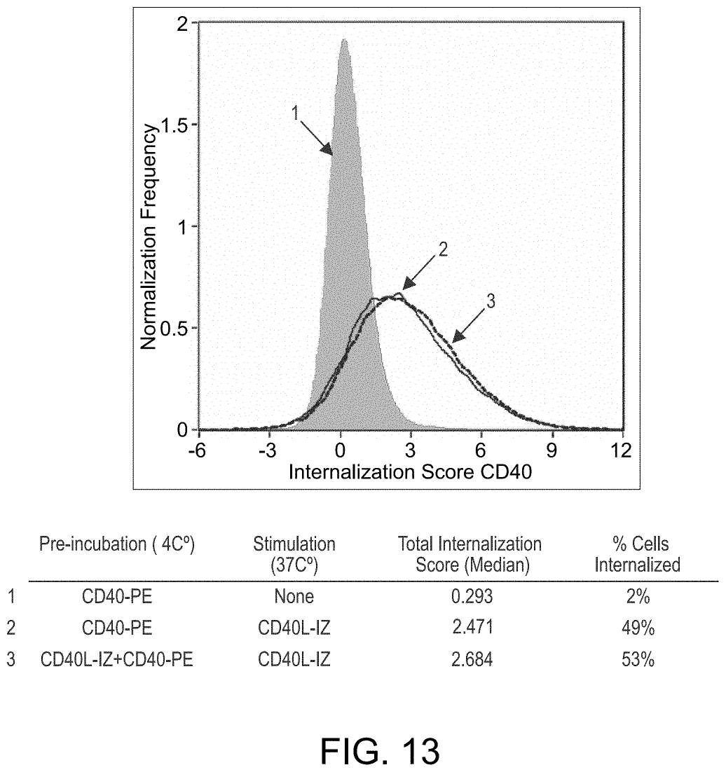

In order to visualize internalization of CD40, Applicants used an Amnis® ImageStream that combines fluorescence microscopy with the throughput and power of quantification of a flow cytometer. Freshly isolated NHV B cells display a regular ring-shaped pattern of CD40 staining on the surface (FIG. 5A). Prior to stimulation, cells were stained with anti-CD40 PE at 4° C. Upon a short stimulation (1 h) with CD40L-IZ at 37° C., the CD40 staining became punctuated, characteristic of aggregation and internalization (FIG. 5B). Internalization was quantified with the Internalization feature [19]. Briefly, it measures the ratio of fluorescence intensity inside the cell (as defined by a 4-pixel erosion of the bright field of the entire cell) to the fluorescence intensity of the entire cell (as defined by the bright field). This ratio is mapped to a log scale, therefore a positive value means medium-to-high internalization whereas a negative value means no-to-low internalization. Unstimulated freshly isolated B cells display an average internalization score of 0.41(FIG. 5D and G). Upon a 1 h stimulation with CD40L-IZ, the average internalization score was increased more than 5 times to 2.09 (p<0.05) (FIG. 5D and G). By contrast, CD45, which is not internalized following CD40L-IZ stimulation, had an average internalization score of 0.67 in unstimulated cells and 0.68 in CD40L-IZ-stimulated B cells (p=1.00) (FIG. 5E and G). Using an internalization score cutoff of 2.5, based on a low frequency of B cells with internalized CD40 in unstimulated sample (2.1%), Applicants determined that 42% of B cells had internalized CD40 after CD40L-IZ stimulation (FIG. 5H). Pre-incubating cells with CD40L-IZ at 4° C., in addition to anti-CD40-PE, did not affect CD40 staining and internalization, showing that CD40L-IZ does not block binding of anti-CD40-PE to CD40 (FIG. 13). Therefore, Applicants confirmed that the rapid loss of CD40 on the surface of B cells following CD40 triggering was due to CD40L-mediated internalization. African American SLE patients had increased expression of surface CD40L concomitant to the lower expression of CD40 on B cells. Applicants then went on to confirm that a membrane form of CD40L could lead to CD40 internalization, using CHO cells stably transfected with human CD40L (hCD40L-CHO). Purified B cells showed downregulation of surface CD40 expression following 1 h co-incubation with hCD40L-CHO cells (FIG. 4B). The extent of surface CD40 downregulation was dependent on the number of hCD40L-CHO cells: 10% of hCD40L-CHO cells led to 37% total CD40^{lo/-} B cells, whereas 1% of hCD40-CHO cells induced CD40 downregulation in only 5% of B cells. CD86 was upregulated in B cells co-cultured with hCD40L-CHO cells at 24 h, confirming their activation (FIG. 4B). The decrease in CD40 surface expression in B cells co-cultured with hCD40L-CHO cells was not transient like in B cells stimulated with CD40L-IZ, likely because of the constitutive expression of CD40L by the CHO cells. CD40 internalization on B cells following co-culture with 10% hCD40L-CHO cells was confirmed by Amnis® ImageStream and was similar to CD40L-IZ stimulation (FIG. 5C, D, G, H) (average internalization score: 1.69 (p<0.05 vs. unstimulated), 33% of cells with CD40 internalization (p<0.05 vs. unstimulated)).

CD40 engagement leads to activation of multiple pathways, including the NF-κB pathway. Applicants therefore quantified the nuclear translocation of the NF-κB p50 sub-unit following CD40 activation using the Similarity feature, which measures the similarity of p50 NF-κB fluorescence to 7-Aminoactinomycin D (7-AAD) nuclear staining [20]. Applicants used a cut-off of similarity>0 for NF-κB nuclear translocation. 29% of unstimulated cells had some degree of nuclear translocation (FIG. 5A, F, I). After stimulation with CD40L-IZ and hCD40L-CHO cells the frequency of cells presenting with p50 nuclear translocation was greatly increased (58% (p<0.05) and 54% (p<0.05), respectively) (FIG. 5B, C, F, I). In conclusion, CD40 stimulation of B cells with CD40L-IZ or hCD40L-CHO cells induced both CD40 internalization and downstream signaling.

### CD40L Upregulated on B Cells Can Trigger CD40 Activation on Adjacent B Cells in a Feed-Forward Loop

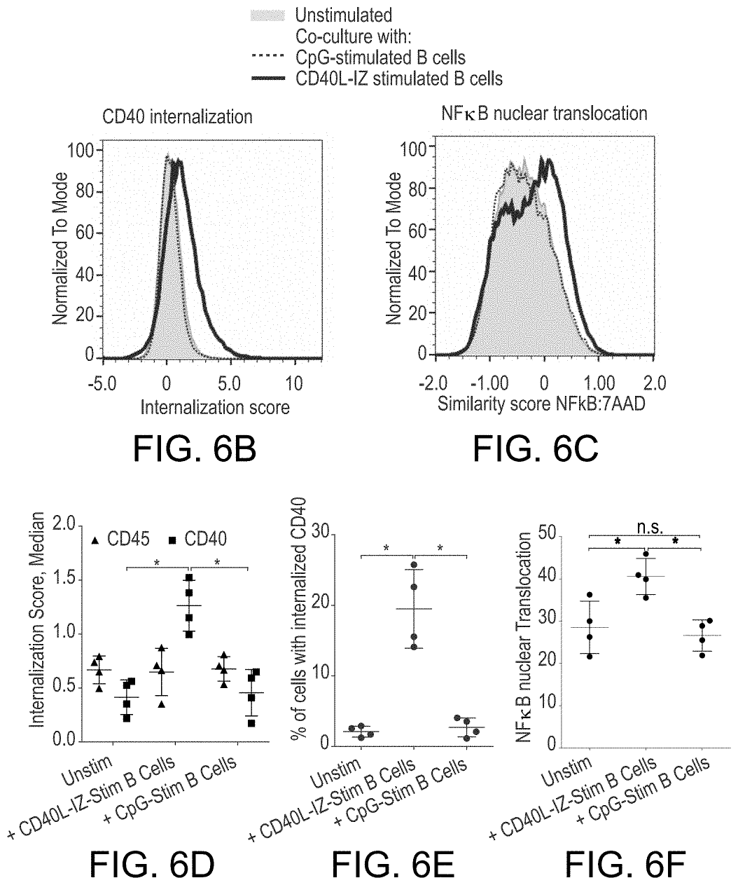

Applicants then sought to induce CD40L expression on B cells. Stimulation for 3 days of purified B cells with CD40L-IZ lead to upregulation of CD40L, concomitant to CD86 upregulation (FIG. 6A). In contrast, B cells stimulated through TLR9 with CpG upregulated CD86 but very little CD40L (FIG. 6A).

Applicants then explored if CD40L upregulated on B cells could induce CD40 internalization and NF-κB translocation. Applicants cultured purified NHV B cells with CD40L-IZ or CpG for 3 days and confirmed CD40L upregulation in the cells cultured with CD40L-IZ. The CpG− and CD40L-IZ-stimulated B cells were the washed and co-cultured with freshly isolated B cells from the same donors at a 1:1 ratio. The fresh B cells were labeled with anti-CD40-PE (to follow CD40 internalization) and with anti-CD45 APC-Cy7, which allowed Applicants to distinguish them from the CpG− or CD40L-IZ-stimulated B cells. After one hour of co-culture, Applicants analyzed CD40 internalization and p50 NF-κB translocation on the CD45-APC-Cy7 labeled B cells. B cells that had been cultured with CD40L-IZ and had upregulated CD40L were able to induce CD40 internalization on freshly isolated autologous B cells (average internalization score of 1.26 vs. 0.41 in unstimulated cells, p<0.05, 19.5% of cells with internalized CD40 vs. 2.1% in unstimulated cells, p<0.05) (FIG. 6B, D, E). By contrast, CpG-stimulated B cells, which only marginally augmented CD40L expression, did not induce CD40 internalization (average internalization score of 0.46, 2.7% of cells with internalized CD40) (FIG. 6B, D, E). Applicants also observed a small increase in p50 nuclear translocation in B cells co-cultured with CD40L-IZ-stimulated B cells (average: 41%) that reached statistical significance (p<0.05). The same was not seen with CpG-stimulated B cells (average: 27%) (FIG. 6F). In conclusion, Applicants demonstrated that upon CD40 triggering, B cells upregulated CD40L, which was able to induce CD40 internalization and activation in trans on adjacent B cells thereby creating a feed-forward loop.

### Increased DN IgD-CD27− B Cell Frequencies in African American SLE Patients

In order to determine if the activated phenotype of B cells from African American SLE patients could potentially result in dysregulated B cell subsets and B-cell driven autoimmunity, Applicants analyzed the frequencies of B cell populations in a subgroup of our cohort that contained 21 African American patients and 21 European American patients. Patients characteristics (disease scores, medications, co-morbidities) were similar in this subgroup and the original cohort (Table 2).

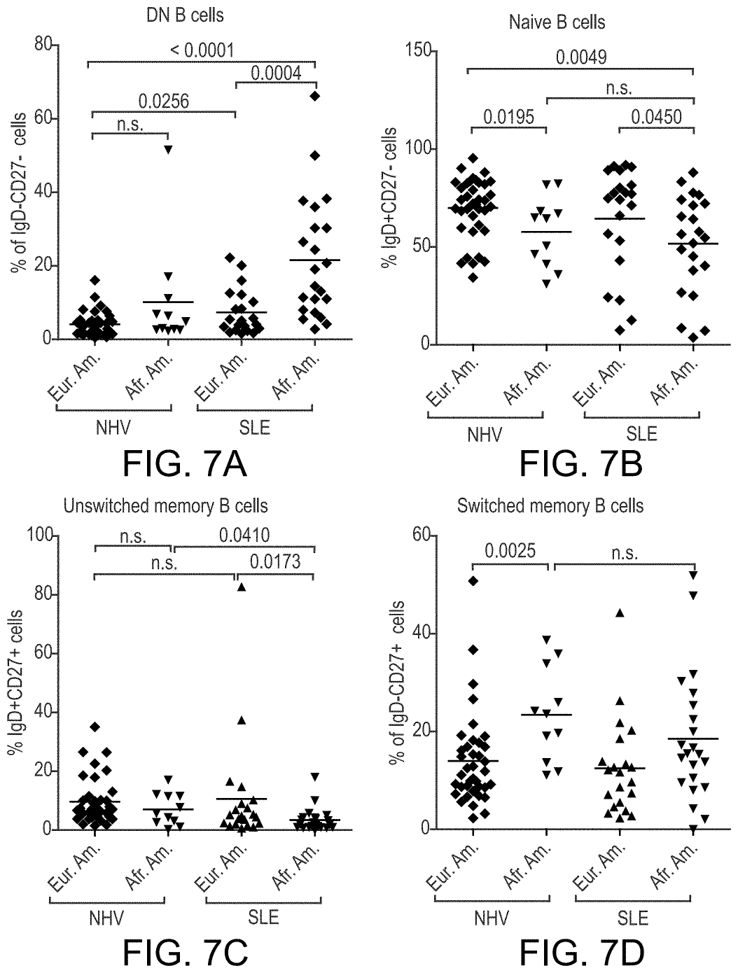

TABLE 2
Clinical data of sub-cohort described in FIG. 7

| | African | European |
|---|---|---|
| | Americans | Americans |
| | (n = 21) | (n = 21) |
| SLEDAI-2K, mean ± SD | 4.2 ± 4.0 | 3.3 ± 1.6 |
| Total ACR classification criteria, mean ± SD | 5.3 ± 1.4 | 5.2 ± 1.3 |
| Duration of disease (years), mean ± SD | 13 ± 7.9 | 17.3 ± 15.5 |
| Time since last flare (years), mean ± SD | 2.6 ± 3.5 | 3.3 ± 5.1 |
| Co-morbidities | | |
| Nephritis, n(%) | 9(43) | 6(29) |
| Sjogren Syndrome, n(%) | 0 | 3(14) |
| Antiphospholipid syndrome, n(%) | 0 | 1(4-8) |
| Medications | | |
| Hydroxychloroquine, n(%) | 10(48) | 9(43) |
| Mycophenolate mofetil, n(%) | 6(29) | 7(33) |
| Belimumab, n(%) | 1(4.8) | 1(4-8) |
| Glucocorticoids, n(%) | 14(67) | 9(43) |

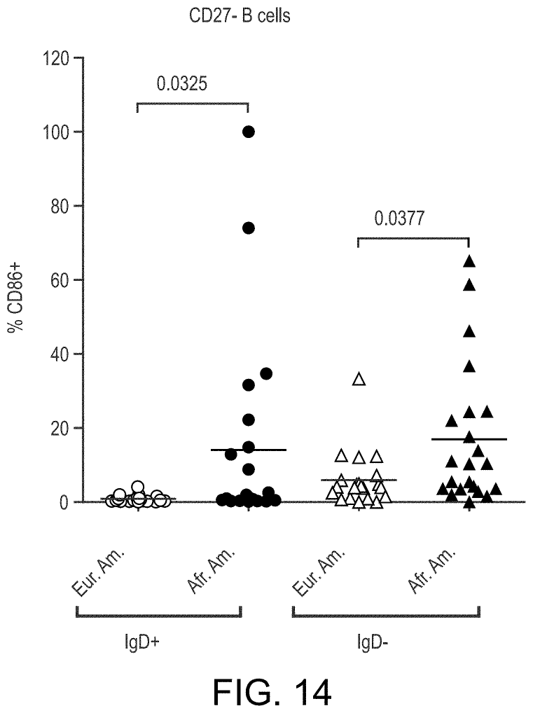

This analysis of cell subset frequencies was performed on whole blood. Applicants found that, in addition to being increased in all SLE patients compared to NHV, as previously reported [21-23], double negative (DN) CD19+CD27−IgD− B cells were greatly enriched in African American patients (FIG. 7A). Doublets and CD3+ T cells were excluded from the B cell subset analysis (FIG. 14). CD19+CD27+IgD+ unswitched memory B cells, on the other hand, were underrepresented in African American patients vs. patients of European ancestry and NHV (FIG. 7C). The frequencies of CD19+CD27+IgD− switched memory B cells were similar in SLE patients and NHV of same ancestries. In fact, switched memory B cells were increased in frequency in African American individuals, regardless whether they were healthy controls or SLE patients (FIG. 7D). Naïve B cell frequencies were reduced in all African American individuals compared to European Americans, with no differences between SLE and NHV (FIG. 7B). Except for a slight decrease of CD4 T cells frequencies in African American patients, other immune cell subsets (monocytes, NK cells, subsets of helper T cells) were not differentially distributed in the two ancestral backgrounds (Table 3).

TABLE 3
Average frequencies of immune cell subsets in SLE patients

| | African | European | |
|---|---|---|---|
| | Americans | Americans | |
| | (n = 21) | (n = 21) | p-value |
| CD19+ B cells, % of WBC | 3.1 ± 3.7 | 3.2 ± 2.4 | p > 0.05 |
| IgD−CD27− (DN) B cells, % of CD19+ cells | 20.2 ± 15.6 | 7.4 ± 6.1 | 0.0012 |
| IgD+CD27− Naïve B cells, % of CD19+ cells | 53 ± 24.9 | 64.4 ± 27 | p > 0.05 |
| IgD−CD27+ switched memory B cells, % of | 19.4 ± 12.9 | 12.5 ± 9.9 | p > 0.05 |
| CD19+ cells | | | |
| IgD+ CD27+ unswitched memory B cells, % of | 3.6 ± 4 | 10.6 ± 18.4 | p > 0.05 |
| CD19+ cells | | | |
| CD19+IgD−CD27^{hi}CD38^{hi} CD2O^{lo} plasmablasts, % | 0.24 ± 0.45 | 0.21 ± 0.53 | p > 0.05 |
| of CD19+ cells | | | |
| CD19+IgD+CD27−CD24^{hi}CD38^{hi} transitional B | 5.7 ± 7.1 | 2.6 ± 3.3 | p > 0.05 |
| cells, % of CD19+ cells | | | |
| CD4 T cells, % of WBC | 8.1 ± 6.9 | 11.2 ± 5.8 | 0.0252 |
| CD8 T cells, % of WBC | 5.6 ± 4.0 | 9.5 ± 13.9 | p > 0.05 |
| CD4-CD8-DN T cells, % WBC | 1.3 ± 2.2 | 1.6 ± 2.3 | p > 0.05 |
| CD3+CD4+CD25+CD127^{lo}Treg, % of CD4+ | 9.6 ± 6.7 | 8.7 ± 7.9 | p > 0.05 |
| T cells | | | |
| CD3+CD56+NKT, % of CD3+ T cells | 9.4 ± 11.3 | 3.6 ± 2.9 | p > 0.05 |
| CD3−CD19−CD20−CD14−CD56+ NK, % of WBC | 1.7 ± 1.4 | 1.8 ± 1.2 | p > 0.05 |
| CD3−CD19−CD20−CD14+ monocytes, % of WBC | 5.3 ± 4.1 | 6.1 ± 4.7 | p > 0.05 |

Data are represented as mean ± SD.
Adjusted p-value < 0.05 (Mann Whitney) are indicated in bold.
WBC: white blood cells;
DN: double negative;
Treg: regulatory T cells,
NKT: natural killer T cells

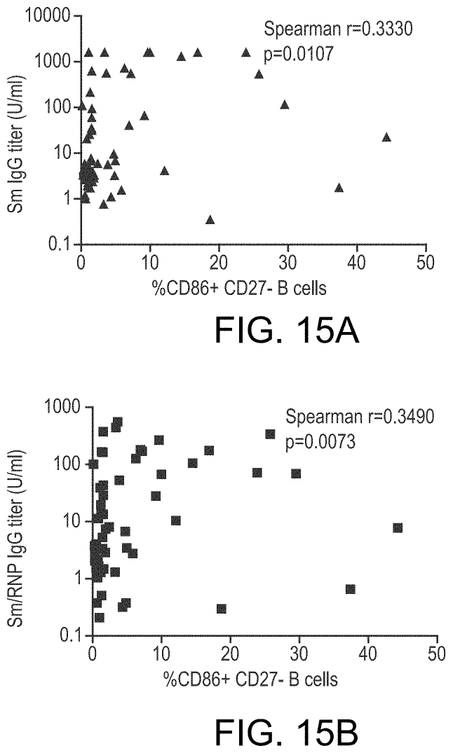

CD27− B cells contain both naïve IgD+ and DN IgD− B cells. IgD+ represent on average 88% and 67% of CD27− B cells in SLE patients of European and African ancestries respectively. To rule out that the increased frequencies of DN B cells in African American patients could explain the increased frequencies of CD86+ CD27− B cells described in FIG. 1, Applicants compared the expression of CD86 by CD27− IgD+ (naïve) and CD27−IgD−(DN) B cells. Even though DN B cells express more CD86 than naïve B cells, both IgD+ and IgD− CD27− B cells displayed an increase in the percent of CD86+ cells in African Americans vs European Americans (FIG. 15).

African Ancestry is the Strongest Factor Associated with the Increased Activated B Cell Phenotype Observed in SLE Patients

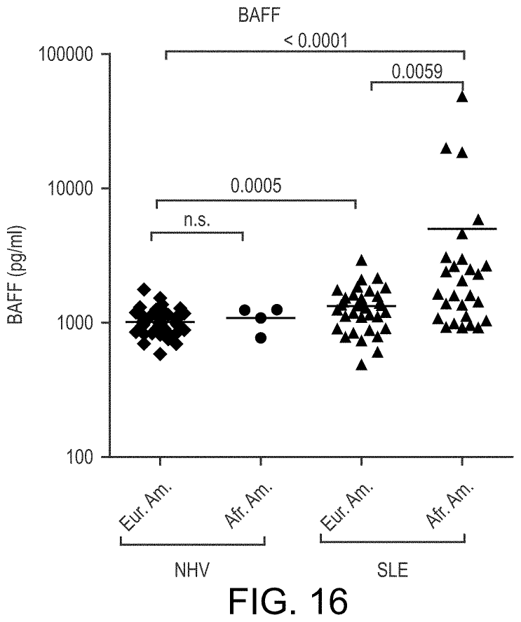

To independently confirm that self-reported African American ancestry was the main factor associated with the differences in B cell phenotypes, rather than other confounding factors such as medication, Applicants performed multiple linear regression analyses over a total of 15 demographic and clinical factors, including sex, age, duration of disease, disease scores, co-morbidities, clinical treatment, etc. (detailed in Methods section). For all six B cell phenotypic endpoints tested as response variables (% of DN, CD86+CD27−, CD86+CD27+, CD40L+CD27−, CD40^{lo}CD27−, CD40^{lo}CD27+ B cells), the African American ancestry was the strongest variable associated (Table 4). Other variables more weakly associated with these parameters include the total count of ACR criteria associated with the percentage of DN B cells, and glucocorticoid use associated with increased percentages of CD86+ CD27− and CD86+ CD27+ B cells. This suggests that glucocorticoid use is linked to the frequencies of CD86+ CD27− and CD86+CD27+ B cells to a lesser extent than African American ancestry. Although glucocorticoids have been previously shown to increase CD40L expression by lymphocytes [24], Applicants could not identify an effect of glucocorticoid use on the frequencies of CD40L+ CD27− B cells in our cohort (FIG. 16). Other medications tested (hydroxychloroquine and mycophenolate mofetil) were not correlated with any of the measured B cell endpoints. Some factors had a negative predictive value, such as duration of disease for the frequencies of CD86+CD27− B cells, and the presence of discoid rash in the African American population for the percentages of CD40L+ CD27− B cells. To conclude, the B cell phenotype observed in African American patients is unlikely due to differences in medication.

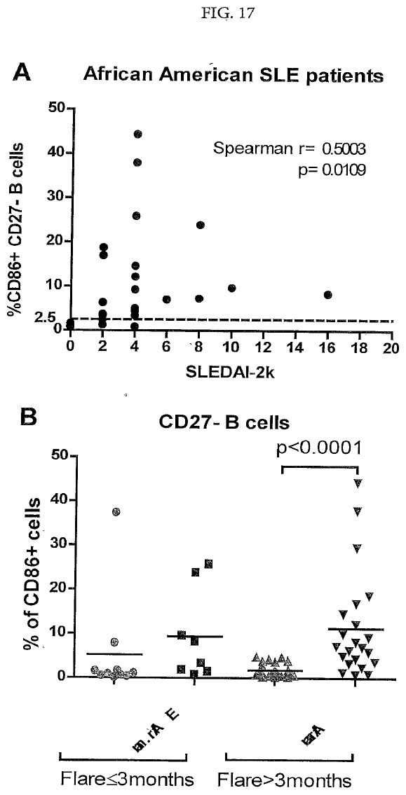

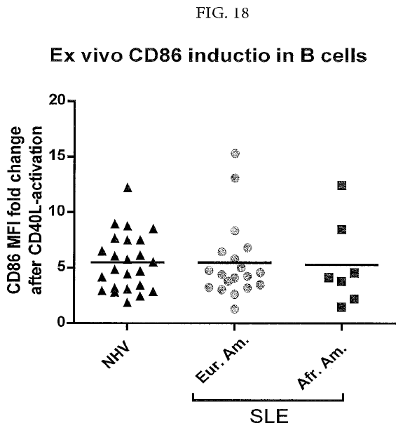

Although SLEDAI-2k was not identified as a confounding factor for the activated B cell phenotype, Applicants observed a moderate correlation between SLEDAI-2k and the percentage of CD86+ CD27− B cells in African American SLE patients (FIG. 17A). To insure that the activated B cell phenotype harbored by these patients was not a result of previous flares, Applicants compared the frequencies of B cells with an activated phenotype in patients who recently flared vs. those who did not, for each ancestral background. Frequencies of CD86+CD27− B cells were similar in patients who flared recently and those who did not (FIG. 17B). Other endpoints (% of DN B cells, % of CD86+ CD27+ B cells, % of CD40^{lo} CD27− and CD27+ B cells, % of CD40L+CD27− B cells) showed similar results (data not shown). Applicants also ruled out the possibility that active LN could drive this phenotype, as only 2/14 African American and 1/15 European American LN patients had active nephritis (other LN patients had inactive nephritis). Therefore, it is unlikely that the activated B cell phenotype that is more pronounced in African American patients is a consequence of recent or current disease activity. Finally, as Applicants were confident that the enrichment of B cells with an activated phenotype in African American patients was not due to other confounding factors, Applicants tested whether B cells from African American SLE patients were more responsive to CD40L stimulation ex vivo. An overnight stimulation of whole blood B cells with CD40L-IZ revealed a similar upregulation of CD86 surface expression in NHV and SLE patients, and in African American and European American SLE patients (FIG. 18). Applicants could therefore not show an intrinsic propensity of African American SLE B cells to respond differently to stimulation through the CD40 pathway.

TABLE 4
African American ethnicity is the strongest predictor for the activated B cell
phenotype of SLE patients.

| | | log (β | |
|---|---|---|---|
| Endpoints | variable | Coefficient) | p-value |
| % DN B cells | Ethnicity (Afr. Am.) | 1.133 | 2.24E−05 |
| | total ACR | 0.344 | 0.0018 |
| % CD86+CD27−B cells | Ethnicity (Afr. Am.) | 1.133 | 6.29E−05 |
| | Glucocorticoids | 0.817 | 0.00987 |
| | Duration of disease | −0.0275 | 0.0126 |
| | Low complement | 0.315 | 0.0181 |
| % CD86+CD27+ B cells | Ethnicity (Afr. Am.) | 0.495 | 0.00623 |
| | Glucocorticoids | 0.373 | 0.0387 |
| % CD40^{lo}CD27− B cells | Ethnicity (Afr. Am.) | 1.455 | 0.00079 |
| % CD40^{lo}CD27+ B cells | Ethnicity (Afr. Am.) | 0.411 | 0.0129 |
| % CD40L+CD27− B cells | Ethnicity (Afr. Am.) | 1.42 | 5.93E−06 |
| | Ethnicity (Afr. Am.):Discoid rash | −1.064 | 0.0263 |

Multiple linear regression analysis was used to evaluate the association for each of 6 indicated response endpoints and 15 demographic and clinical endpoints as co-variates.
Only co-variates displaying statistical significance (p-value < 0.05) are shown.
Afr. Am.: African American

Patients with Higher CD40^{lo} CD27− B Cell Frequencies have also Increased Anti-Sm, Anti-Sm/RNP and Anti-dsDNA Autoantibody Titers

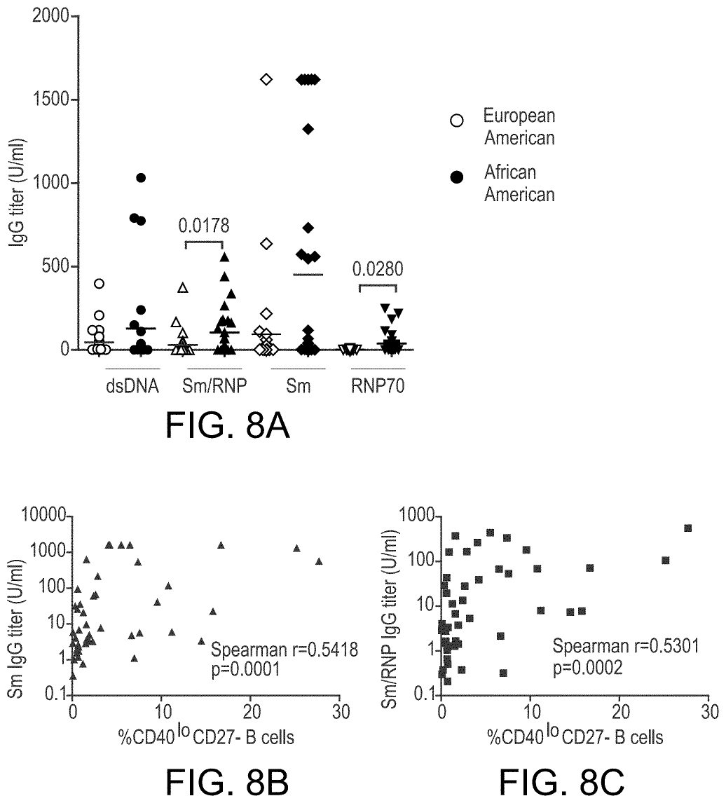

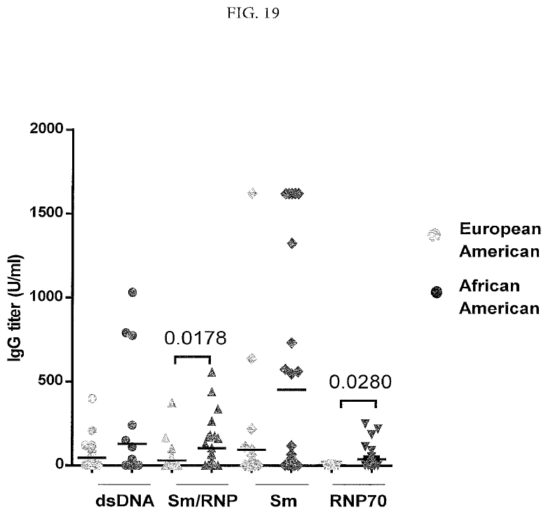

The secretion of autoantibodies is a hallmark of SLE. By forming immune complexes with autoantigens, autoantibodies have a direct pathogenic role on tissues and organs, and activate innate and adaptive immune cells. In fact, the presence of autoantibodies, such as anti-dsDNA antibodies, has been associated with flares [6, 25]. Therefore, Applicants analyzed antibody titers in African American patients vs. patients of European descent. There was an increase of anti-Sm/RNP and anti-RNP70 titers in African Americans SLE patients compared to European American patients (FIG. 19). Anti-Sm autoantibodies were also increased in African American patients, compared to patients of European descent, but the difference did not reach statistical significance (FIG. 19). Applicants then inquired whether patients with higher frequencies of CD40^{lo} B cells also had higher titers of autoantibodies, analyzing European American and African American patients separately. African Americans SLE patients with CD40^{lo} CD27− B cells frequencies higher than 1.54%, which corresponds to the 90^{th} percentile of CD40^{lo} CD27− B cell frequencies in NHV, had significantly higher anti-Sm/RNP, anti-Sm and anti-dsDNA IgG plasma levels. In addition, European American patients with higher anti-Sm/RNP, anti-Sm and anti-dsDNA titers also had higher frequencies of CD40^{lo} CD27− B cells, the difference reaching significance for anti-Sm titers (FIG. 8). These results support the hypothesis that the particular B cell phenotype observed in SLE African American patients reflects an increased activation of B cells, possibly via the CD40 pathway.

### Methods

### Patients

Applicants obtained peripheral blood in 2014 and 2015 from 68 SLE patients (29 patients self-identified as ‘Black or African American’ and 39 patients of European ancestry self-identified as ‘Caucasian’) who were visiting their physician at Northwell Health, Great Neck, N.Y. Most patients were on standard of care treatment for general SLE. Details of medication and associated co-morbidities are summarized in Table 1. Healthy subjects were analyzed in parallel (Table 5). Blood was shipped overnight. Immediately upon reception, plasma was collected and frozen for further use and peripheral blood mononuclear cells (PBMC) were purified.

TABLE 5
Comparison of patients' and controls' demographics

| | SLE (n = 68) | NHV(n = 69) |
|---|---|---|
| Age(years), mean ± SD | 46 ± 15 | 45 ± 12 |
| Female, n (%) | 57(84) | 53(77) |
| African American ethnicity, n (%): | 29(43) | 13(19) |

### Flow Cytometry

80 μl of heparin anticoagulated blood or 1 million freshly isolated PBMC were incubated with pre-mixed cocktails of conjugated antibodies. Antibodies used for whole blood were: CD3-eFluor®450 or CD3-Alexa Fluor(AF)700 (both OKT3), CD45RA-fluorescein isothiocyanate (FITC) (JS-83), CD27− allophycocyanin (APC) (O323), IgD-FITC (IA6-2), CD24− phycoerythrin (PE) (SN3 A5-2H10), CD38-peridinin-Chlorophyll-protein(PerCP)-eFluor710(HB7) (all eBiosciences), CD4-PE-cyanine(Cy)7(OKT4), CXCR3-AF647(G025H7), CCR6-Brilliant violet(BV)785(G034E3), PD1-BV605(EH12.2H7), CD19-BV421 (HIB19) (all Biolegend), CD8a-APC-H7(SK1), CCR7-PE-CF594(150503), CXCR5-BV510(RF8B2), CD20-APC-H7 (2H7) (all BD Biosciences); for PBMC: CD3-APC-eFLuor®780(UCHT1), CD4-PerCPCy5.5(RPA-T4), CD8a-PECy7(RPA-T8), PD1-PerCPCy5.5(EH12.2H7), (all eBiosciences), CD19-APC-Cy7(HIB19), CD40-PE(5C3), CD40L-PE(24-31), CD80-FITC(2D10), CD86-APC(IT2.2), CD45RO-Pacific Blue (UCHL1) (all Biolegend), CD27-BV605(L128) (BD Biosciences). Whole blood samples were lysed for red blood cells and fixed with FACS lysing buffer. Stained PBMC samples were fixed in 1.5% paraformaldehyde. Samples were run on LSR-Fortessa or LSRII (BD Biosciences) and analyzed with FlowJo V10.0.7. Exclusion of doublets was systematically applied in the gating strategy (FIG. 14).

Upregulation of Surface Markers During in vitro B Cell Activation

Total B cells were purified from freshly isolated PBMC by magnetic negative selection as described by manufacturer (Stemcell tech.). 250,000 cells/well were cultured in RPMI supplemented with antibiotics and 10% FBS without or with 1 μg/ml of human CD40L isoleucine zipper (CD40L-IZ) [26], 20 μg/ml of goat anti-human IgM F(ab′)₂ (Jackson Immunoresearch), 1 μg/ml of CpG ODN2006-B (Invivogen) or co-cultured with 25,000 Chinese hamster ovary (CHO) cells stably transfected with human CD40LG (hCD40L-CHO cells) at Bristol-Myers Squibb. Parental CHO DG44 cells were obtained from Dr. Lawrence Chasin (Columbia University, New York, N.Y.). At indicated timepoints, cells were collected, washed and stained with CD19-APC-Cy7(HIB19, Biolegend), CD27-BV605(L128, BD Biosciences), CD80-FITC(2D10, Biolegend), CD86-APC(IT2.2, Biolegend), CD40-PE, (5C3, Biolegend), CD40L-PE(24-31 Biolegend), PD1-PerCPCy5.5 (EH12.2H7, eBiosciences), fixed in 1% paraformaldehyde and run on LSRII (BD Biosciences). Samples were analyzed with FlowJo V10.0.7.

To compare the response to CD40L stimulation by B cells from different ancestral backgrounds, CD40L-driven upregulation was tested in a whole blood assay. Ninety μl of heparin anticoagulated blood (SLE and NHV) was rested for one hour before addition of 10 μg/ml of CD40L-IZ. After an overnight incubation, samples were stained with CD20-APC and CD86-PE (eBiosciences) and run on Canto II (BD Biosciences).

### CD40 Receptor Internalization and NF-κB Nuclear Translocation

Total B cells were isolated from frozen PBMCs by magnetic negative selection as described by manufacturer (StemCell Technologies, Inc.). Freshly isolated B cells were stained for 30 min on ice with anti-CD40-PE (clone 5C3, BioLegend) and with anti-CD45-APC/Cy7 (clone H130, BioLegend) in Stain Buffer (BSA) (BD Biosciences) containing Hu FcR Binding Inhibitor (eBioscience). After staining, B cells (3.0×10⁶ cells/well in 12 well plate) were incubated for 1 h at 37° C. (5% CO₂) in RPMI 1640 supplemented with heat-inactivated 10% FBS, 1% penicillin-streptomycin and 1% L-glutamine in the presence or absence of 1 μg/ml CD40L-IZ, hCD40L-CHO cells (1:10 hCD40L-CHO: B cell ratio), or autologous B cells previously activated for 72 hrs at 37° C. (5% CO₂) in RPMI with 1 μg/ml CD40L-IZ or 1 μg/ml CpG ODN2006-B (Invivogen) (1:1 ratio). B cell stimulation was stopped by incubating cells on ice for 10 minutes. Cells were washed and stained on ice in BD Stain Buffer with CD19-BV510 (clone H1B19, BioLegend). After fixation in 4% paraformaldehyde (PFA) (Alfa Aesar), cells were permeabilized for 20 min at 4° C. in 1X BD Perm/Wash buffer (BD Biosciences). Permeabilized cells were then stained on ice in Perm/Wash buffer with anti-NF-κB p50-AF488 (clone 4D1, BioLegend), washed in Perm/Wash buffer, and then fixed again. A nuclear staining dye, 7-AAD Viability Staining Solution (BioLegend) was added to all samples ten minutes prior to data acquisition.

### ImageStream Data Acquisition and Analysis

ImageStream data acquisition and analysis were performed as previously described [19, 20]. Data acquisition was done using Amnis® ImageStream^{X} Mark II imaging flow cytometer (EMD Millipore) and INSIRE acquisition software. Collected images were analyzed using IDEAS V.6.2 image-analysis software (Amnis/EMD Millipore). In each sample, sixty thousand events were collected and imaged in the Extended Depth of Field mode (EDF). Digital spectral compensation was performed on a pixel-by-pixel basis using single-stained controls. Acquired cellular imagery was analyzed for the degree of CD40 and CD45 internalization using the Internalization feature [19], and for the degree of NF-κB p50 nuclear translocation using the Similarity feature [20], as described in IDEAS V.6.2 documentation.

### Enzyme-Linked Immunosorbent Assay (ELISA)

Levels of sCD40L and BAFF in plasma were detected with human CD40L and human BAFF ELISA kits respectively (both R&D Systems) following manufacturer instructions. For autoantibody titers, plasma samples were diluted 100 fold into sample dilution buffer and incubated on a pre-coated plate with dsDNA (ALPCO), Sm (ALPCO), Sm/RNP (ALPCO) or RNP70 (Genway). ELISA was developed with horseradish peroxidase conjugated anti-human IgG followed by TMB substrate. The reaction was stopped with 1M Hydrochloric acid and read on dual wavelength spectrophotometer. Values were calculated based on the standard curve and were reported as IU/ml.

### Statistics

Descriptive and single factor statistical analyses were performed with GraphPad Prism 5. Mann-Whitney non parametric T test was used to compare groups. P values were adjusted to correct for multiple comparisons and repeated measures. The following formula was used: adjusted p=1−(1−α)^{k}, where α is the non-adjusted p value, and k is the number of comparisons. k was set at 30. Correlations were analyzed with Spearman correlation. P value of 0.05 or lower was considered significant.

Multiple linear regression analysis was performed in R statistical package. To ensure normality, log transformation was used for all six tested endpoints as response variables (% of CD86+ in CD27− and CD27+ B cells, % of CD40^{lo} CD27− and CD27+ B cells, % of CD40L+ CD27− B cells, % of DNB cells). The co-variates tested as predictor variables were: age, sex, self-reported African American ancestry, duration of disease, SLEDAI-2k, total count of ACR criteria and some of its components (renal disorder, discoid rash, malar rash and arthritis), the presence of nephritis and low complement and treatment with hydroxychloroquine, mycophenolate mofetil and glucocorticoids. The presence of the co-morbidities ITP, Sjogren's syndrome, antiphospholipid syndrome and the effect of belimumab were not tested because of the paucity of samples being positive for these variables. A variable selection procedure based on Akaike's information criterion was used to select informative predictor variables.

### References

1. Mohan C, Putterman C. Genetics and pathogenesis of systemic lupus erythematosus and lupus nephritis. Nat Rev Nephrol. 2015;11(6):329-341.

2. Jacobi AM, Odendahl M, Reiter K, et al. Correlation between circulating CD27high plasma cells and disease activity in patients with systemic lupus erythematosus. Arthritis Rheum. 2003;48(5):1332-1342.

3. Gonzalez LA, Toloza SM, McGwin G, Jr., Alarcon GS. Ethnicity in systemic lupus erythematosus (SLE): its influence on susceptibility and outcomes. Lupus. 2013;22(12):1214-1224.

4. Sule S, Fivush B, Neu A, Furth S. Increased risk of death in African American patients with end-stage renal disease secondary to lupus. Clin Kidney J. 2014;7(1):40-44.

5. Nee R, Martinez-Osorio J, Yuan CM, et al. Survival Disparity of African American Versus Non-African American Patients With ESRD Due to SLE. Am J Kidney Dis. 2015.

6. Petri M, Singh S, Tesfasyone H, Malik A. Prevalence of flare and influence of demographic and serologic factors on flare risk in systemic lupus erythematosus: a prospective study. J Rheumatol. 2009;36(11):2476-2480.

7. Merrill JT, Neuwelt CM, Wallace DJ, et al. Efficacy and safety of rituximab in moderately-to-severely active systemic lupus erythematosus: the randomized, double-blind, phase II/III systemic lupus erythematosus evaluation of rituximab trial. Arthritis Rheum. 2010;62(1):222-233.

8. Rovin BH, Furie R, Latinis K, et al. Efficacy and safety of rituximab in patients with active proliferative lupus nephritis: the Lupus Nephritis Assessment with Rituximab study. Arthritis Rheum. 2012;64(4):1215-1226.

9. Bijl M, Horst G, Limburg PC, Kallenberg CG. Expression of costimulatory molecules on peripheral blood lymphocytes of patients with systemic lupus erythematosus. Ann Rheum Dis. 2001;60(5):523-526.

10. Agata Y, Kawasaki A, Nishimura H, et al. Expression of the PD-1 antigen on the surface of stimulated mouse T and B lymphocytes. Int Immunol. 1996;8(5):765-772.

11. Koshy M, Berger D, Crow MK. Increased expression of CD40 ligand on systemic lupus erythematosus lymphocytes. J Clin Invest. 1996;98(3):826-837.

12. Desai-Mehta A, Lu L, Ramsey-Goldman R, Datta SK. Hyperexpression of CD40 ligand by B and T cells in human lupus and its role in pathogenic autoantibody production. J Clin Invest. 1996;97(9):2063-2073.

13. Manea ME, Mueller RB, Dejica D, et al. Increased expression of CD154 and FAS in SLE patients' lymphocytes. Rheumatol Int. 2009;30(2):181-185.

14. Vakkalanka RK, Woo C, Kirou KA, Koshy M, Berger D, Crow MK. Elevated levels and functional capacity of soluble CD40 ligand in systemic lupus erythematosus sera. Arthritis Rheum. 1999;42(5):871-881.

15. Grammer AC, Bergman MC, Miura Y, Fujita K, Davis LS, Lipsky PE. The CD40 ligand expressed by human B cells costimulates B cell responses. J Immunol. 1995;154(10):4996-5010.

16. Manning E, Pullen SS, Souza DJ, Kehry M, Noelle RJ. Cellular responses to murine CD40 in a mouse B cell line may be TRAF dependent or independent. Eur J Immunol. 2002;32(1):39-49.

17. Hostager BS, Catlett IM, Bishop GA. Recruitment of CD40 and tumor necrosis factor receptor-associated factors 2 and 3 to membrane microdomains during CD40 signaling. J Biol Chem. 2000;275(20):15392-15398.

18. Wang HM, Yan Q, Yang T, et al. Scaffold protein JLP is critical for CD40 signaling in B lymphocytes. J Biol Chem. 2015;290(9):5256-5266.

19. Vallhov H, Gutzeit C, Johansson SM, et al. Exosomes containing glycoprotein 350 released by EBV-transformed B cells selectively target B cells through CD21 and block EBV infection in vitro. J Immunol. 2011;186(1):73-82.

20. George TC, Fanning SL, Fitzgerald-Bocarsly P, et al. Quantitative measurement of nuclear translocation events using similarity analysis of multispectral cellular images obtained in flow. J Immunol Methods. 2006;311(1-2):117-129.

21. Simpson N, Gatenby PA, Wilson A, et al. Expansion of circulating T cells resembling follicular helper T cells is a fixed phenotype that identifies a subset of severe systemic lupus erythematosus. Arthritis Rheum. 2010;62(1):234-244.

22. Choi JY, Ho JH, Pasoto SG, et al. Circulating follicular helper-like T cells in systemic lupus erythematosus: association with disease activity. Arthritis Rheumatol. 2015;67(4):988-999.

23. Huang W, Sinha J, Newman J, et al. The effect of anti-CD40 ligand antibody on B cells in human systemic lupus erythematosus. Arthritis Rheum. 2002;46(6):1554-1562.

24. Jabara HH, Brodeur SR, Geha RS. Glucocorticoids upregulate CD40 ligand expression and induce CD40L-dependent immunoglobulin isotype switching. J Clin Invest. 2001;107(3):371-378.

25. Petri MA, van Vollenhoven RF, Buyon J, et al. Baseline predictors of systemic lupus erythematosus flares: data from the combined placebo groups in the phase III belimumab trials. Arthritis Rheum. 2013;65(8):2143-2153.

26. Sharma S, Jin Z, Rosenzweig E, Rao S, Ko K, Niewold TB. Widely divergent transcriptional patterns between SLE patients of different ancestral backgrounds in sorted immune cell populations. J Autoimmun. 2015;60:51-58.

27. Ritterhouse LL, Crowe SR, Niewold TB, et al. B lymphocyte stimulator levels in systemic lupus erythematosus: higher circulating levels in African American patients and increased production after influenza vaccination in patients with low baseline levels. Arthritis Rheum. 2011;63(12):3931-3941.

28. Qamar N, Fuleihan RL. The hyper IgM syndromes. Clin Rev Allergy Immunol. 2014;46(2):120-130.

29. Han S, Hathcock K, Zheng B, Kepler TB, Hodes R, Kelsoe G. Cellular interaction in germinal centers. Roles of CD40 ligand and B7-2 in established germinal centers. J Immunol. 1995;155(2):556-567.

30. Bolduc A, Long E, Stapler D, et al. Constitutive CD40L expression on B cells prematurely terminates germinal center response and leads to augmented plasma cell production in T cell areas. J Immunol. 2010;185(1):220-230.

31. Higuchi T, Aiba Y, Nomura T, et al. Cutting Edge: Ectopic expression of CD40 ligand on B cells induces lupus-like autoimmune disease. J Immunol. 2002;168(1):9-12.

32. Tipton CM, Fucile CF, Darce J, et al. Diversity, cellular origin and autoreactivity of antibody-secreting cell population expansions in acute systemic lupus erythematosus. Nat Immunol. 2015;16(7):755-765.

33. Wei C, Anolik J, Cappione A, et al. A new population of cells lacking expression of CD27 represents a notable component of the B cell memory compartment in systemic lupus erythematosus. J Immunol. 2007;178(10):6624-6633.

34. Wu YC, Kipling D, Dunn-Walters DK. The relationship between CD27 negative and positive B cell populations in human peripheral blood. Front Immunol. 2011;2:81.

35. Fecteau JF, Cote G, Neron S. A new memory CD27−IgG+ B cell population in peripheral blood expressing VH genes with low frequency of somatic mutation. J Immunol. 2006;177(6):3728-3736.

36. Bagnara D, Squillario M, Kipling D, et al. A Reassessment of IgM Memory Subsets in Humans. J Immunol. 2015;195(8):3716-3724.

37. Morbach H, Wiegering V, Richl P, et al. Activated memory B cells may function as antigen-presenting cells in the joints of children with juvenile idiopathic arthritis. Arthritis Rheum. 2011;63(11):3458-3466.

38. Yurasov S, Wardemann H, Hammersen J, et al. Defective B cell tolerance checkpoints in systemic lupus erythematosus. J Exp Med. 2005;201(5):703-711.

39. Isnardi I, Ng YS, Menard L, et al. Complement receptor 2/CD21− human naive B cells contain mostly autoreactive unresponsive clones. Blood. 2010;115(24):5026-5036.

40. Eris JM, Basten A, Brink R, Doherty K, Kehry MR, Hodgkin PD. Anergic self-reactive B cells present self antigen and respond normally to CD40-dependent T-cell signals but are defective in antigen-receptor-mediated functions. Proc Natl Acad Sci U S A. 1994;91(10):4392-4396.

41. Jacobi AM, Huang W, Wang T, et al. Effect of long-term belimumab treatment on B cells in systemic lupus erythematosus: extension of a phase II, double-blind, placebo-controlled, dose-ranging study. Arthritis Rheum. 2010;62(1):201-210.

42. Lodolce JP, Kolodziej LE, Rhee L, et al. African-derived genetic polymorphisms in TNFAIP3 mediate risk for autoimmunity. J Immunol. 2010;184(12):7001-7009.

43. Tavares RM, Turer EE, Liu CL, et al. The ubiquitin modifying enzyme A20 restricts B cell survival and prevents autoimmunity. Immunity. 2010;33(2):181-191.

44. Chen JM, Guo J, Wei CD, et al. The association of CD40 polymorphisms with CD40 serum levels and risk of systemic lupus erythematosus. BMC Genet. 2015;16(1):121.

45. Vazgiourakis VM, Zervou MI, Choulaki C, et al. A common SNP in the CD40 region is associated with systemic lupus erythematosus and correlates with altered CD40 expression: implications for the pathogenesis. Ann Rheum Dis. 2011;70(12):2184-2190.

46. Katsiari CG, Liossis SN, Souliotis VL, Dimopoulos AM, Manoussakis MN, Sfikakis PP. Aberrant expression of the costimulatory molecule CD40 ligand on monocytes from patients with systemic lupus erythematosus. Clin Immunol. 2002;103(1):54-62.

47. Matsuura JE, Morris AE, Ketchem RR, et al. Biophysical characterization of a soluble CD40 ligand (CD154) coiled-coil trimer: evidence of a reversible acid-denatured molten globule. Arch Biochem Biophys. 2001;392(2):208-218.

We claim:

1. A method of treating or preventing lupus in a subject, comprising:
(a) identifying the subject as having at least one differentially regulated biomarker selected from CD40, CD40L, CD86, CD80, and PD1; and
(b) administering an agent that inhibits the CD40 or CD28 signaling pathway,
thereby treating or preventing lupus in the subject.

2. A method of treating or preventing lupus in a subject, comprising:
(a) administering an agent that inhibits the CD40 or CD28 signaling pathway;
(b) determining whether the agent neutralizes at least one differentially regulated biomarker selected from CD40, CD40L, CD86, CD80, and PD1; and
(c) adjusting the dosing of the agent in the subject,
thereby treating or preventing lupus in the subject.

3. The method of claim 1 or 2, wherein the differentially regulated biomarker comprises down-regulated expression of CD40.

4. The method of claim 1 or 2, wherein the differentially regulated biomarker comprises up-regulated expression of CD40L.

5. The method of claim 1 or 2, wherein the differentially regulated biomarker comprises up-regulated expression of CD86.

6. The method of claim 1 or 2, wherein the differentially regulated biomarker comprises up-regulated expression of CD80.

7. The method of claim 1 or 2, wherein the differentially regulated biomarker comprises up-regulated expression of PD1.

8. The method of claim 1, comprising identifying the subject as having at least two differentially regulated biomarker selected from CD40, CD40L, CD86, CD80, and PD1.

9. The method of claim 8, comprising identifying the subject as having at least three differentially regulated biomarker selected from CD40, CD40L, CD86, CD80, and PD1.

10. The method of claim 2, comprising determining whether the agent neutralizes at least two differentially regulated biomarker selected from CD40, CD40L, CD86, CD80, and PD1.

11. The method of claim 10, comprising determining whether the agent neutralizes at least three differentially regulated biomarker selected from CD40, CD40L, CD86, CD80, and PD1.

12. The method of claim 1 or 2, wherein the agent specifically binds to CD40, CD40L, or CD28.

13. The method of claim 12, wherein the agent is selected from an anti-CD40 antibody, an anti-CD40L antibody, and an anti-CD28 antibody.

14. The method of claim 1 or 2, wherein the lupus is systemic lupus erythematosus (SLE).

15. A kit comprising: (1) an antibody which specifically binds to at least one differentially regulated biomarker selected from CD40, CD40L, CD86, CD80, and PD1; and (2) instructions for use of said kit.
